(19) United States
(12) Patent Application Publication
(10) Pub. No.: US 2022/0265254 A1
(43) Pub. Date: Aug. 25, 2022

(54) POLYURETHANE-REINFORCED HYDROGEL CARDIAC PATCH

(71) Applicant: The Regents of the University of Colorado, a Body Corporate, Denver, CO (US)

(72) Inventors: Jeffrey G. Jacot, Aurora, CO (US); Zei-wei Tao, Aurora, CO (US); Anne C. Lyons, Aurora, CO (US); Emily C. Beck, Aurora, CO (US)

(21) Appl. No.: 17/634,676

(22) PCT Filed: Aug. 13, 2020
(86) PCT No.: PCT/US2020/046231

§ 371 Date: Feb. 11, 2022

Related U.S. Application Data
(60) Provisional application No. 62/886,795, filed on Aug. 14, 2019.

(51) Int. Cl. A61B 17/00; A61L 27/38; A61L 27/48; A61L 27/52; A61L 27/54; A61L 27/58

(52) U.S. Cl. CPC A61B 17/0057; A61L 27/3808; A61L 27/48; A61L 27/52; A61L 27/54; A61L 27/58; A61B 2017/00004; A61B 2017/00243; A61B 2017/00884; A61L 2430/20

(57) ABSTRACT

Disclosed herein are devices and methods for repairing a heart defect. The disclosed devices, comprising a biodegradable gel and a biodegradable mesh scaffold, enhance cellular infiltration, vascularization, and degredation, while reducing fibrosis and rejection. In many embodiments, the mesh scaffold comprises one or more of polycaprolactone, gelatin, and polyurethane, and the gel comprises a biologically active compound decorated with polyethylene glycol. The disclosed heart patch devices possess elasticity and strength similar existing patch products derived from mammalian pericardium.

### FIELD

The disclosed processes, methods, and devices are directed to a repairing defects in mammalian tissue, for example a cardiac patch device for repairing heart defects.

### CROSS-REFERENCE TO RELATED APPLICATIONS

This application claims benefit of priority pursuant to 35 U.S.C. § 119(e) of U.S. provisional patent application No. 62/886,795 entitled “POLYURETHANE-REINFORCED HYDROGEL CARDIAC PATCH,” filed on 14 Aug. 2020, which is hereby incorporated by reference in its entirety.

### BACKGROUND

Congenital heart defects (CHD) affect 1 of every 111 to 125 births in the United States. An estimated 40,000 infants are affected by CHD each year; of these, about 25% require invasive treatment in the first year of life. Surgical repair of CHD often requires the use of a polymer or fixed tissue patch to close septal defects or enlarge stenosed structures. Approximately 50% of Tetralogy of Fallot repairs include a patch in the right ventricular (RV) outflow tract. Currently, surgeons use synthetic or biological materials, including knitted polyethylene terephthalate (most commonly Dacron®), expanded polytetrafluoroethylene (such as Gore-Tex®), and glutaraldehyde-fixed bovine pericardium (such as SJ Medical and CardioCel®). These materials do not grow with the pediatric patients, are not electromechanically integrated, have mismatched mechanical properties compared with the surrounding tissue, and often become fibrotic, leading to an increased risk of malicious arrhythmia, sudden cardiac death, and heart failure. With the conventional patches discussed above, about 25% of patch-implanted patients require a second surgery.

The information included in this Background section of the specification, including any references cited herein and any description or discussion thereof, is included for technical reference purposes only and is not to be regarded subject matter by which the scope of the invention as defined in the claims is to be bound.

### SUMMARY

Disclosed herein are heart/cardiac patch devices useful in correcting various heart defects, as well as methods of manufacturing and methods of using same. The heart patch device of the present disclosure may be used to replace missing or damaged mammalian myocardium, wherein the devices provide for sufficient mechanical integrity, strength, electro- and bio-compatibility to support tissue repair, remodeling, and regeneration. In one embodiment, the disclosed cardiac patch device comprises a flexible but strong biodegradable polymeric scaffold capable of maintaining integrity, and a gel that enhances vascularization and cell infiltration. In many embodiments, the device may be seeded with a plurality of differentiated or non-differentiated cells, the cells may be autologous or non-autologous cells.

This disclosed device is capable of controlled degradation to allow sufficient time for the patient's cells and extracellular matrix to replace the degrading patch. The disclosed device may also be sufficiently porous to help initiate greater vascularization and tissue repair/remodeling while limiting the body's rejection, which may be in the form of fibrosis and/or a fibrotic response. The tendency of the disclosed devices to reduce fibrosis may result in fewer incidences of arrhythmia during repair/regeneration, and greater heart function compared with commercially available patches, e.g. those made of glutaraldehyde-crosslinked bovine pericardium, polyethylene terephthalate, and polytetrafluoroethylene.

Disclosed herein is a heart patch device comprising, a biodegradable polymeric mesh scaffold, and a biodegradable gel. In some embodiments, the the polymeric mesh scaffold may be made of a material comprising one or more of gelatin, polyurethane, and polycaprolactone, and the gel may comprises one or more of fibrin and polyethylene glycol. In many embodiments the device may further comprise one or more mammalian cells, that may be autologous cells and/or non-autologous cells. In most embodiments, where the device comprises mammalian cells, the cells may express one or more of CD31 (PECAM1), CD144 (VE-Cadherin), and CD309 (VEGFR2).

Also disclosed are methods for making a heart patch device, comprising forming a mesh scaffold by electrospinning, forming a gel layer, and combining the mesh scaffold with the gel layer. In some embodiments, the device may comprise an additional gel that may be added to the mesh scaffold after it is positioned adjacent the gel layer. In many embodiments the method may further comprise adding one or more mammalian cells to the device, wherein the cells may be autologous cells and/or non-autologous cells. In most embodiments, where mammalian cells are added to the device, the cells may express one or more of CD31 (PECAM1), CD144 (VE-Cadherin), and CD309 (VEGFR2). In embodiments wherein cells are added to the device, the device may be incubated for 1 or more days.

Also disclosed is a method of treating a subject having a heart defect, the method comprising, obtaining a heart patch device comprising a mesh scaffold and a biodegradable gel, placing the heart patch device at or near the heart defect, connecting the heart patch device to the heart with one or more sutures, and thereby treating the subject having the heart defect. In many embodiments the treatment may further comprise adding one or more mammalian cells to the device prior to implantation, wherein the cells may be autologous cells and/or non-autologous cells. In most embodiments, where mammalian cells are added to the device, the cells may express one or more of CD31 (PECAM1), CD144 (VE-Cadherin), and CD309 (VEGFR2). In embodiments wherein cells are added to the device, the device may be incubated for 1 or more days prior to implantation.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments and implementations and illustrated in the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 Panel A shows a graph of a Fourier transform infrared (FTIR) spectrum of biodegradable poly(ether ester urethane) urea (BPUR).

FIG. 1 Panel B shows a table of peak assignments of BPUR.

FIG. 1 Panel C shows an image of a BPUR reinforcement layer cut from an electrospun mesh.

FIG. 1 Panel D shows a scanning electron microscopy image of an electrospun BPUR.

FIG. 1 Panel E shows an image of a BPUR-reinforced fibrin gel patch.

FIG. 1 Panel F shows a graph of Young's modulus.

FIG. 1 Panel G shows a graph depicting a frequency spectrum of fibrin gel.

FIG. 2 Panel A1 shows an image of a purse string suture created on a RV free wall.

FIG. 2 Panel A2 shows an image of a full-thickness defect from excising the distend portion.

FIG. 2 Panel A3 shows an image of a cardiac patch stitched over the defect of FIG. 2A2.

FIG. 2 Panel A4 shows an image of the purse string suture of FIG. 2A1 released from the RV free wall.

FIG. 2 Panel B1 shows an image of the excised portion of FIG. 2A2.

FIG. 2 Panel B2 shows the image of FIG. 2B1 zoomed in to show the defect.

FIG. 3 shows graphs depicting an ECG arrhythmia recorded for 30 min at 4- and 8-weeks post surgery.

FIG. 4 Panel A1 shows an M-mode image from the parasternal short-axis view of a heart from the sham control group.

FIG. 4 Panel A2 shows a B-mode image from the parasternal long-axis view indicating the LV end-diastolic area (LVEDA) of the heart of FIG. 4A1.

FIG. 4 Panel A3 shows a B-mode image from the parasternal long-axis view indicating the LV end-systolic area of the heart of FIG. 4A1.

FIG. 4 Panel B1 shows an M-mode image from the parasternal short-axis view of a heart from the pericardium group.

FIG. 4 Panel B2 shows a B-mode image from the parasternal long-axis view indicating the LV end-diastolic area (LVEDA) of the heart of FIG. 4B1.

FIG. 4 Panel B3 shows a B-mode image from the parasternal long-axis view indicating the LV end-systolic area of the heart of FIG. 4B1.

FIG. 4 Panel C1 shows an M-mode image from the parasternal short-axis view of a heart from the BPUR PEG-fibrin group.

FIG. 4 Panel C2 shows a B-mode image from the parasternal long-axis view indicating the LV end-diastolic area (LVEDA) of the heart of FIG. 4C1.

FIG. 4 Panel C3 shows a B-mode image from the parasternal long-axis view indicating the LV end-systolic area of the heart of FIG. 4C1.

FIG. 4 Panel D shows a graph depicting changes in the LV end-diastolic area (LVEDA) post surgery.

FIG. 4 Panel E shows a graph depicting changes in the LV end-diastolic dimensions/diameter (LVEDd) post surgery.

FIG. 4 Panel F shows a graph depicting changes in the LV ejection fraction (LVEF) post surgery.

FIG. 4 Panel G shows a graph depicting changes in the LV fractional shortening (LVFS) post surgery.

FIG. 5 Panel A1 shows a graph depicting an RV pressure-volume (PV) loop at 4 weeks post surgery for a heart in the sham control group.

FIG. 5 Panel A2 shows a graph depicting an RV PV loop at 4 weeks post surgery for a heart in the pericardium group.

FIG. 5 Panel A3 shows a graph depicting an RV PV loop at 4 weeks post surgery for a heart in the BPUR PEG-fibrin group.

FIGS. 5 Panel B11shows a graph depicting an RV PV loop at 8 weeks post surgery for a heart in the sham control group.

FIGS. 5 Panel B2 shows a graph depicting an RV PV loop at 8 weeks post surgery for a heart in the pericardium group.

FIGS. 5 Panel B3 shows a graph depicting an RV PV loop at 8 weeks post surgery for a heart in the BPUR PEG-fibrin group.

FIG. 5 Panel C shows a graph depicting right ventricular systolic pressure (RVSP) post surgery.

FIG. 5 Panel D shows a graph depicting right ventricular end-diastolic volume (RVEDV) post surgery.

FIG. 5 Panel E shows a graph depicting right ventricular ejection fraction (RVEF) post surgery.

FIG. 5 Panel F shows a graph depicting right ventricular stroke work (RV SW) post surgery.

FIG. 5 Panel G shows a graph depicting right ventricular cardiac output (RV CO) post surgery.

FIGS. 6 Panels A1-A5 show macroscopic images of the patch area on the RV wall for a heart in the sham control group, the pericardium group at 4- and 8-weeks post surgery, and the BPUR PEG-fibrin group at 4- and 8-weeks post surgery, respectively.

FIG. 6 Panels B1-B5 show images of Masson's trichrome staining depicting patch implantation-induced fibrosis on sections directly through the center of the defect for a heart in the sham control group, the pericardium group at 4- and 8-weeks post surgery, and the BPUR PEG-fibrin group at 4- and 8-weeks post surgery, respectively.

FIGS. 6 Panels C1-C5 show images of insets depicting the degradation of patch materials and defect thickness for a heart in the sham control group, the pericardium group at 4- and 8-weeks post surgery, and the BPUR PEG-fibrin group at 4- and 8-weeks post surgery, respectively.

FIGS. 6 Panels Dl-D5 show images depicting immunofluorescence staining showing filament actin (F-actin positive), cardiac fibroblasts and endothelial cells (vimentin positive), and nuclei (DAPI) for a heart in the sham control group, the pericardium group at 4- and 8-weeks post surgery, and the BPUR PEG-fibrin group at 4- and 8-weeks post surgery, respectively.

FIG. 6E shows a graph depicting changes of patch implantation-induced fibrosis for hearts in the pericardium group and BPUR PEG-fibrin group at 4- and 8-weeks post surgery.

FIG. 6F shows a graph depicting patch material remaining for hearts in the pericardium group and BPUR PEG-fibrin group at 4- and 8-weeks post surgery.

FIG. 6G shows a graph depicting wall thickness for hearts in the sham control group, the pericardium group and the BPUR PEG-fibrin group at 4- and 8-weeks post surgery.

FIG. 6H shows a graph depicting vimentin-positive cell volume for hearts in the pericardium group and BPUR PEG-fibrin group at 4- and 8-weeks post surgery.

FIGS. 7 Panels A1-A5 show 200× magnification images stitched together of an explanted patch stained for α-actinin, αSMA, vWF and DAPI for a heart in the sham control group, the pericardium group at 4- and 8-weeks post surgery, and the BPUR PEG-fibrin group at 4- and 8-weeks post surgery, respectively.

FIGS. 7 Panels B1-B5 show images of vWF staining indicating blood vessels for a heart in the sham control group, the pericardium group at 4- and 8-weeks post surgery, and the BPUR PEG-fibrin group at 4- and 8-weeks post surgery, respectively.

FIGS. 7 Panels C1-05 show images of CD45 staining indicating leukocytes for a heart in the sham control group, the pericardium group at 4- and 8-weeks post surgery, and the BPUR PEG-fibrin group at 4- and 8-weeks post surgery, respectively.

FIGS. 7 Panels D1-D5 show images of CD68 staining indicating pan-macrophages for a heart in the sham control group, the pericardium group at 4- and 8-weeks post surgery, and the BPUR PEG-fibrin group at 4- and 8-weeks post surgery, respectively.

FIGS. 7 Panels E1-E5 show images of CD206 staining indicating M2 macrophages for a heart in the sham control group, the pericardium group at 4- and 8-weeks post surgery, and the BPUR PEG-fibrin group at 4- and 8-weeks post surgery, respectively.

FIG. 7 Panel F shows a graph depicting the number of blood vessels for hearts in the sham control group, the pericardium group and the BPUR PEG-fibrin group at 4- and 8-weeks post surgery.

FIG. 7 Panel G shows a graph depicting the amount of CD45 expression indicating leukocytes for hearts in the pericardium group and the BPUR PEG-fibrin group at 4- and 8-weeks post surgery.

FIG. 7 Panel H shows a graph depicting the amount of CD68 expression indicating pan-macrophages for hearts in the pericardium group and the BPUR PEG-fibrin group at 4- and 8-weeks post surgery.

FIG. 7 Panel I shows a graph indicating the amount of CD208 expression indicating M2 macrophages for hearts in the pericardium group and the BPUR PEG-fibrin group at 4- and 8-weeks post surgery.

FIG. 7 Panel J shows a graph indicating a percentage of M2 over Pan-macrophages for hearts in the pericardium group and the BPUR PEG-fibrin group at 4- and 8-weeks post surgery.

FIGS. 8 Panels Al -A2 show images of an external scar size and internal defect size at 4- and 8-weeks post surgery for a heart in the sham control group.

FIGS. 8 Panels B1-B2 show images of an external scar size and internal defect size at 4- and 8-weeks post surgery for a heart in the pericardium group.

FIGS. 8 Panels C1-C2 show images of an external scar size and internal defect size at 4- and 8-weeks post surgery for a heart in the BPUR PEG-fibrin group.

FIG. 8 Panels D shows a graph depicting the external scar size for hearts in the pericardium group and BPUR PEG-fibrin group at 4- and 8-weeks post surgery.

FIG. 8 Panels E shows a graph depicting the internal defect size for hearts in the pericardium group and BPUR PEG-fibrin group at 4- and 8-weeks post surgery.

FIG. 9 Panel A shows a graph depicting changes in Young's Modulus with the addition of PEO to the scaffold.

FIG. 9 Panel B shows a graph depicting changes in ultimate tensile stress with the addition of PEO to the scaffold.

FIG. 9 Panel C shows a graph depicting changes in porosity with the addition of PEO to the scaffold.

FIG. 9 Panel D shows a graph depicting changes in cell depth with the addition of PEO to the scaffold.

FIG. 10 shows patch fabrication. (Panels A and B) c-Kit+amniotic fluid stem cells (AFSCs c-Kit+) at passage 3 and fluorescence-activated cell sorting (FACS) showing 17.99% c-Kit p positive before plating; (Panel C) Green fluorescent protein expressing human umbilical vein endothelial cells (GFP-HUVECs) at passage 7 before plating; (Panels D and E) Punched biodegradable poly(ether ester urethane) urea (BPUR) support layer and its SEM image; (Panels F and G) BPUR reinforced PEG-fibrin patch and the SEM image showing the integration between the fibrin gel and the mesh layer; (Panels H-J) Formation of GFP-HUVEC branches at day 3 after coculture with c-Kit+AFSCs in a PEG-fibrin gel; (Panel K) Frequency spectrum of PEG-fibrin gel showing the storage and loss moduli.

FIG. 11 shows changes in left ventricular (LV) function at 1- and 2-months post-surgery. (Panels A-C) M and B-mode images show LV chamber in endsystole and end-diastole; (Panels D-G) Graphs show LV end-diastolic diameter (LVEDd), LV ejection fraction (LVEF), and LV fractional shortening (LVFS). Values are mean ±standard deviation. *p<0.05, **p<0.01.

FIG. 12 shows implantation-induced fibrosis, patch degradation, and muscularization at 2 months post-surgery. (Panels A1-A3) Patches on the right ventricle wall; (Panels B1-B3) Implantation-induced fibrosis; (Panels C1-C3) Patch material remaining and muscularization; (D1-3) Vimentin positive cells show granulation tissue. (Panels E-H) Graphs show changes of implantationinduced fibrosis, patch material remaining, muscularization and vimentin-positive cell volume. Values are mean ±standard deviation. *p<0.05, **p<0.01.

FIG. 13 shows blood vessels, α-actinin volume and macrophage infiltration at 2 months post-surgery. (Panels A1-A3) α-Smooth muscle actin (α-SMA) staining shows arterioles and venules; (Panels B1-B3) von Willebrand factor (vWF) staining for counting capillaries; (Panels C1-C3) α-Actinin staining shows muscularization; (Panels D1-D3) CD68 staining shows pan-macrophage infiltration; (Panels E1-E3) CD208 staining shows M2 macrophage infiltration. (Panels F-J) Graphs show α-SMA volume, number of capillaries, α-actinin volume, M2 macrophage infiltration, and percentage of M2 over panmacrophages. Values are mean±standard deviation. *p<0.05, **p<0.01.

### DETAILED DESCRIPTION

This disclosure is related to cardiac implants for correcting heart defects and methods of manufacturing such implants. An implant of the present disclosure may be used to replace heart tissue such as myocardium. The disclosed implants are sufficiently flexible, porous, and strong to provide a stable environment for new cell growth/invasion during repair/regeneration. Thus, the disclosed implants are suitable for use in non-heart tissues and organs, for example muscle tissue, and organs such as diaphragm, bladder, and uterus. In some cases the implants may be used to aid in wound repair. The disclosed devices provide mechanical integrity, biocompatibility, and support for repair and regeneration of missing or damaged tissue. In many embodiments, the disclosed biodegradable polymer-reinforced hydrogel heart/cardiac patch device is designed to maintain its structural integrity it degrades to allow the body's cells and secreted extracellular matrix to repopulate the defect or injury. The device is also designed to initiate and support great vascularization while limiting the body's rejection, in the form of fibrosis. The disclosed heart patch device may result in fewer incidences of arrhythmia, during repair/remodeling, as well as provide for greater heart function compared with commercially available natural and synthetic patches. In most embodiments, the disclosed cardiac patch device possesses mechanical properties approximating native tissue, allowing it to more closely resemble the performance of native tissue.

### Uses of Device

The presently implant device may be used in various tissues for repair and/or remodeling of the tissue. In many embodiments, the device may be useful in repairing congenital defects. In other embodiments, the disclosed device may be useful in repair of injuries such as hernias, for example diaphragmatic hernias. In other embodiments, the disclosed device may be useful in repair of injuries such as wounds, especially large wounds.

### Patch Composition and Size

The presently disclosed heart patch device may include a gel portion and a porous mesh scaffold. In many embodiments, the gel may comprise one or more bioactive, biodegradeable materials, and the porous mesh framework may be manufactured of one or more biodegradable polymeric materials, in one example biodegradable polyurethane (BPUR). In many embodiments, the gel portion may be one or more discrete layers and/or may be contained within the porous mesh scaffold. In some embodiments, heart patch device may include a gel layer and a porous mesh scaffold layer adjacent the gel layer. In some embodiments, additional gel material may be contained within the mesh scaffold layer.

The disclosed cardiac patch device may be of various sizes and shapes. In some embodiments, the cardiac patch device may be sized while it is fabricated, and in other embodiments it may be reduced in size after it is fabricated, for example by cutting or trimming. Adjusting the size of the heart patch device may aid in it being readily degradable and may facilitate its integration into mammalian tissue.

The disclosed implant patch may have various shapes. In many embodiments, the shape of the implant device may be modified by a medical professional prior to implantation. In some embodiments, the device may be, for example, generally round, oval, square, rectangular or irregularly shaped. In many embodiments, the implant device may define an average diameter of about 0.5-5 cm, and an average thickness of about 0.05-2 cm. Average diameter is not meant to limit the shape or size of patches to round objects, rather all shapes (e.g. squares, ovals, triangles, etc.) may define an average diameter.

### Mesh Scaffold

The disclosed mesh scaffold may be constructed using various methods. In some embodiments, the mesh scaffold is constructed by one or more methods that produce a porous material that is greater than 60% porous, as measured gavimetrically. For example, the porosity of the mesh scaffold may be greater than about 65%, 70%, 72%, 74%, 76%, 78%, 80%, 82%, 84%, 86%, 88%, 90%, 91%, 92%, 93%, 94%, 95%, 96%, 97%, 98%, or 99%, and less than about 100%, 99%, 98%, 97%, 96%, 95%, 94%, 93%, 92%, 91%, 90%, 89%, 88%, 87%, 86%, 85%, 80%, 75%, 70%, or 65%. In some embodiments the mesh framework may be constructed from a method that creates an irregular or regular pattern. In one embodiment, the method of manufacture may be electrospinning. In other embodiments, the method of manufacture may be selected from 3D printing, additive manufacturing, molding, spin coating, electrospinning, and combinations thereof. The disclosed mesh scaffold may have an average thickness between about 20 μm and 400 μm, for example greater than about 20 μm, 30 μm, 40 μm, 50 μm, 60 μm, 70 μm, 80 μm, 90 μm, 100 μm, 110 μm, 120 μm, 130 μm, 140 μm, 150 μm, 200 μm, 250 μm, 300 μm, or 350 μm, and less than about 4000 μm, 350 μm, 300 μm, 250 μm, 200 μm, 150 μm, 140 μm, 130 μm, 120 μm, 110 μm, 100 μm, 90 μm, 80 μm, 70 μm, 60 μm, 50 μm, 40 μm, and 30 μm.

One or more nanoparticles may be incorporated into the mesh scaffold during fabrication to help regulate the size and number of pores. In most embodiments, the nanoparticle is removable, for example dissolvable or degradable. In one embodiment, the nanoparticle is composed of polyethylene oxide (PEO).

The mesh scaffold may be trimmed and/or shaped after fabrication. In many embodiments, for example wherein the mesh is electrospun, the edges of the mesh may be manually trimmed to remove edges and/or to attain a desired shape. In some embodiments, channels through the mesh scaffold may be created after fabrication, for example by inserting a needle or other object through the scaffold. In some embodiments, the channels are formed by extracting material from the fabricated scaffold.

The polymeric material of the mesh scaffold may be biodegradeable, elastic, and with a tensile strength suitable for resisting stresses in a target tissue. In many embodiments, that target tissue is cardiac tissue, and the suitable tensile strength is greater than about 100 kPa. In various embodiments, the disclosed mesh scaffold may have an average Young's Modulous of less than about 50 Mpa, 45 MPa, 40 MPa, 35 MPa, 30 MPa, 25 MPa, 20 MPa, 15 MPa, 10 MPa, 5 MPa, 4 MPa, 3 MPa, 2 MPa, 1 MPa, 0.9 MPa, 0.8 MPa, 0.7 MPa, 0.6 MPa, 0.5 MPa, 0.4 MPa, 0.3 MPa, 0.2 MPa, 0.1 MPa, 0.09 MPa, 0.05 MPa, or 0.02 MPa, and greater than about 0.01 MPa, 0.02 MPa, 0.03 MPa, 0.04 MPa, 0.05 MPa, 0.06 MPa, 0.07 MPa, 0.08 MPa, 0.09 MPa, 0.1 MPa, 0.2 MPa, 0.3 MPa, 0.4 MPa, 0.5 MPa, 1.0 MPa, 1.5 MPa, 2 MPa, 3 MPa, 4 MPa, 5 MPa, 10 MPa, 15 MPa, 20 MPa, 25 MPa, 30 MPa, 35 MPa, 40 MPa, or 45 MPa. In many embodiments, the mesh scaffold material may comprise one or more natural and/or synthetic compounds, for example, proteins, polymeric proteins, monomers, polymers, diols, diisocyanates, esters, ethers, urethanes, gelatins, lactones, etc. In some embodiments, the material may be selected from one or more of poly lactone, polycaprolactone, poly urethane, poly urethane urea, poly (ether-ether) urethane, poly (ether-ester) urethane, and poly (ether-ester) urethane urea. In many embodiments, the material may comprise a polymer having a ‘hard segment,’ rigid group,' or ‘hard group,’ and a non-hard segment or linker. In some embodiments, the hard group may comprise about 5-50% of the polymer, for example 10% of the polymer. The amount of hard segment may be varied to alter the performance characteristics (such as stiffness, elasticity, degradability, etc.) of the polymer.

### Gel

The gel may be comprised of various compounds, for example natural and/or synthetic compounds that degrade in the mammalian body, support vascularization, and cellular reconstruction. In one embodiment, the gel may comprise a mammalian clotting agent, such as fibrin. The compounds may be combined with other compounds to aid in stability, vascularization, etc. In one embodiment the compounds may be covalently decorated with polyethylene glycol (PEG), for example PEGylated human fibrinogen. The disclosed gel may be in the form of a layer, having a thickness between about 800 μm to 2 mm, for example greater than about 810 μm, 820 μm, 850 μm, 900 μm, 950 μm, 1000 μm, 1100 μm, 1200 μm, 1300 μm, 1400 μm, 1500 μm, 1600 μm, 1700 μm, 1800 μm, or 1900 μm, and less than about 2000 μm, 1900 μm, 1800 μm, 1700 μm, 1600 μm, 1500 μm, 1400 μm, 1300 μm, 1200 μm, 1100 μm, 1000 μm, 950 μm, 900 μm, and 850 μm.

The gel may be formed by allowing the gel to solidify in a mold of various shapes and sized. In many embodiments the gel may be formed between plates of a given separation distance. The gel may compressed after formation by about 5% to 40%, for example more than about 5%, 6%, 7%, 8%, 9%, 10%, 11%, 12%, 13%, 14%, 15%, 16%, 17%, 18%, 19%, 20%, 21%, 22%, 23%, 24%, 25%, 30%, or 35%, and less than about 40%, 35%, 30%, 25%, 24%, 23%, 22%, 21%, 20%, 19%, 18%, 17%, 16%, 15%, 14%, 13%, 12%, 11%, 10%, 9%, 8%, 7%, or 6%.

### Methods of Fabricating

The presently disclosed patches may be fabricated by various methods. In one embodiment, the patches may be reinforced fibrin-containing patches. In some embodiments, the patches are fabricated by mixing PEGylated human fibrinogen with human thrombin plus. In some embodiments, one or more cells may be mixed with the fibrinogen and thrombin, for example stem cells and or endothelial cells, such as human umbilical vein endothelial cells (HUVECs) or amniotic fluid stem cells (AFSCs). In various embodiments the patch may be reinforced with one or more BPUR layers. In many embodiments, the cell-impregnated patches may be cultured in a tissue culture environment in various appropriate media. In one embodiment, the media may be GEM-2 media. The patch may be cultured for various periods, for example from about 0-24 hours or 1-14 days, for example 3 days, before the patch is implanted.

### Seeding Cells

The disclosed devices could be seeded with a combination of endothelial cells and/or support cells. In some embodiments, seeding may be useful to enhance vascularization and other aspects of the device's integration. The seeded cells could be autologous or provided from matched donors and/or cells. Seeded cells could be differentiated or undifferentiated. In many embodiments, seeded cells may be positive for CD31 (PECAM1), CD144 (VE-Cadherin), and CD309 (VEGFR2) and may be obtained from amniotic fluid, umbilical cord, adult stem cells, or induced pluripotent stem cells. The seeded cells may be endothelial cells that may be sorted or verified positive for one or more surface markers, for example one or more of. In some embodiments, the seed cells may be one or more of fractionated or unfractionated mesenchymal stem cells, general fibroblasts, smooth muscle cells, etc. The seeded cells may be combined and seeded into the patch along with the PEG-fibrin. These patches could be implanted immediately, or cultured for up to 2 weeks to allow for microvessel formation, and then implanted. In some embodiments the seeded cells may be c-Kit+ cells, for example stem cells and/or endothelial cells, for example umbilical epithelial cells. In various embodiments, the cells may be c-Kit+ cells from amniotic fluid.

### Exemplary Patch Benefits

Disclosed herein is an improved implant device, with beneficial characteristics. In some embodiments the implant device is a cardiac/heart patch device. The disclosed heart patch device may comprise an angiogenic polymer, for example a poly(ethylene glycol) fibrin-biodegradable hydrogel, and may be reinforced with polymeric scaffold, for example an electrospun biodegradable poly(ether ester urethane) urea (BPUR) mesh layer. The disclosed implant device may enhance cell invasion, angiogenesis, and regenerative remodeling. The disclosed heart patch device may aid in improving defect closure, cardiac output, and heart function as measured by echocardiogram, electrocardiogram, and pressure loop measurements. In many embodiments, effectiveness of the implant device may be measured by analyzing extent of fibrosis, macrophage infiltration, vascularization, etc. post-implantation. Compared with traditional fixed pericardium patches, the disclosed reinforced hydrogel patches result in fewer arrhythmias and greater ventricular ejection fraction, fractional shorting, stroke work, and cardiac output. Further, implanted patches of the present disclosure may degrade at a higher rate. Less of the disclosed implant device was shown to remain in the body at 4- and 8-weeks post-implantation, as compared to conventional patches.

### EXAMPLES

### Example 1—Patch Fabrication

Experiments were performed to create a cardiac patch with reduced induction of fibrosis and improved vascularization. Previous research indicated that right ventricular (RV) wall replacement with a multi-layered patch composed of a chitosan-gelatin-heart matrix hydrogel reinforced with a polycaprolactone (PCL) membrane resulted in higher RV ejection fractions compared with fixed bovine pericardium at 8 weeks post surgery. However, the multi-layered patch induced significant fibrosis in the RV wall and relatively poor vascularization. In order to increase vascularization, a gel composed of fibrin covalently decorated with poly(ethylene glycol) (PEG) was developed and tested in a subcutaneous mouse model. The disclosed gel induced rapid gel vascularization with increased scaffold stability compared with fibrin alone. In addition, biodegradable polyurethanes with hydrolytically or enzymatically cleavable moieties were selected as an alternative to PCL, which takes 2-3 years to resorb in vivo, to reduce fibrosis.

A myocardial replacement patch of PEG-fibrin reinforced with an electrospun poly(ether ester urethane) urea mesh layer was fabricated. This engineered cardiac patch was tested in an RV wall replacement model in adult rats and compared with a sham surgery control and a clinical control of glutaraldehyde-fixed pericardium. Heart function was measured at 4- and 8-weeks post surgery, and histologic sections were evaluated for fibrosis, macrophage infiltration, vascularization, and defect size.

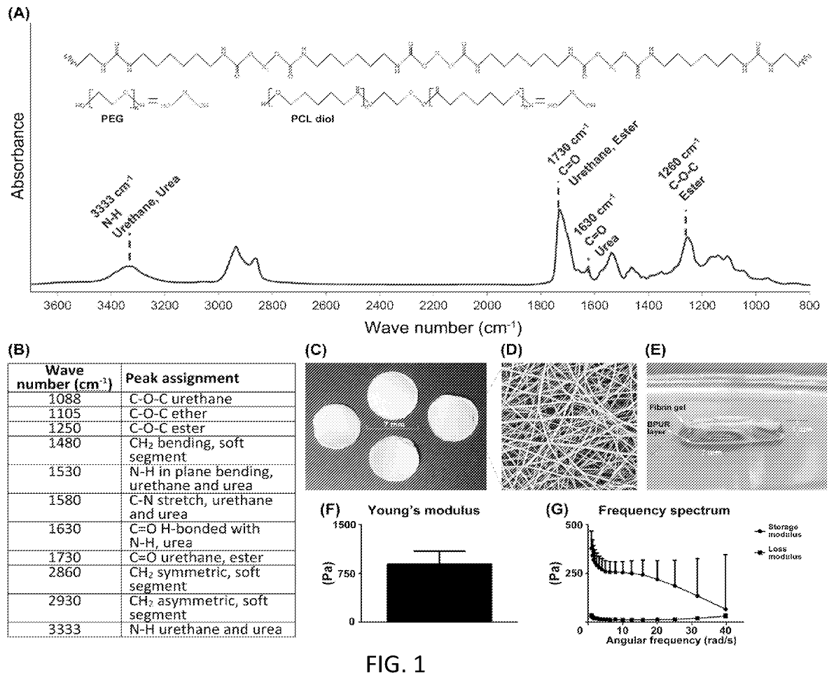

A biodegradable poly(ether ester urethane) urea (BPUR) with 10% hard segment was synthesized, for example, as described in A. P. Kishan, T. Wilems, S. Mohiuddin, E. M. Cosgriff-Hernandez, Synthesis and Characterization of Plug-and-Play Polyurethane Urea Elastomers as Biodegradable Matrixes for Tissue Engineering Applications, ACS Biomaterials Science & Engineering 3(12) (2017) 3493-502, which is hereby incorporated by reference herein in its entirety. For example, a poly(ether ester) triblock was synthesized by reacting poly(ethylene glycol) diisocyanate (PEG-DI) and polycaprolactone (PCL, MW=530 Da). PCL with stannous octoate (0.1 wt % with respect to the polymer) was added dropwise into a flask containing PEG-DI to a final PEG-DI:PCL molar ratio of 1:2 under nitrogen with stirring at 80° C. for 7 hours. BPUR was then synthesized in a two-step process from this triblock diol and hexane diisocyanate (HDI) using ethylene diamine (ED) as a chain extender at a molar ratio of 1:2:1 triblock diol:HDI:ED. A 10 wt % solution of the triblock diol in N,N-dimethylformamide (DMF) containing 0.1 wt % stannous octoate was first added dropwise to a flask containing a 10 wt % solution of HDI in DMF under nitrogen. The reaction proceeded at 80° C. under a nitrogen blanket with constant stirring until no change in the hydroxyl stretch was observed via transmission Fourier transform infrared (FTIR) spectroscopy (about 5 hours). The reaction was then cooled to room temperature. Chain extension was then performed by adding a 10 wt % solution of ED in DMF dropwise to the prepolymer solution under vigorous stirring. The BPUR chemical structure was confirmed using transmission FTIR spectroscopy. Neat BPUR films were cast onto KBr pellets from 1,1,1,3,3,3-hexafluoro-2-propanol (HFIP, Sigma, St. Louis, Mo.) solutions (5 wt %) and placed under vacuum for 1 hour under ambient conditions to remove the solvent. Spectra were recorded using a Nicolet iS10 (Thermo Scientific) FTIR spectrometer at a resolution of 2 cm-1 for 64 scans. Full reaction was confirmed by absence of the isocyanate peak at 2267 cm-1 (FIG. 1A). Peaks at 3333 cm-1 (N-H stretch) and 1630 cm-1 (C=O stretch of ordered C=O . . . H-N in the urea group) indicated successful chain extension. Whole peak assignments were shown in FIG. 1B. These results were consistent with previous infrared spectral analysis.

A 10 wt % solution of BPUR in HFIP was used to electrospin fibrous meshes. BPUR solutions were dispensed using a syringe pump at a constant rate of 0.3 ml/hour. A positive voltage of 7.5 kV was applied at the needle tip, which was placed 17 cm from a −5-kV charged copper plate. Electrospinning was performed at ambient conditions (25° C., 45-55% relative humidity). The resulting fabricated electrospun meshes were vacuum dried for a minimum of 12 hours prior to characterization. The average thickness of BPUR meshes was 80±10 μm (n=5) (FIG. 1C). The fiber morphology was characterized using scanning electron microscopy (Phenom Pro, NanoScience Instruments, Phoenix, Ariz.) at 10 kV accelerating voltage. Specimens were cut from the center of each fiber mesh to avoid edge effects. Prior to scanning electron microscopy imaging, the specimens were coated with 4 nm of gold using a sputter coater (Sputter Coater 108, Cressingtion Scientific Instruments, Hertfordshire, UK). BPUR fiber meshes displayed an average fiber diameter of 1.5±0.8 μm (n=5) (FIG. 1 D). Finally, tensile testing of electrospun BPUR meshes were performed on dogbone specimens cut in accordance with ASTM D1708 and strained to failure at a rate of 100%/min using an Instron 3345 uniaxial tensile tester equipped with a 100 N load cell and pneumatic side action grips (Instron 2712-019). A secant modulus based on 2% strain was calculated for the elastic modulus from the resultant engineering stress-strain plots (n=5). BPUR meshes had an average tensile modulus of 2.9±0.4 MPa (n=5).

BPUR support layers (7 mm in diameter) were cut from the electrospun BPUR mesh. To enhance gel integration with the mesh, 5 evenly spaced holes were made with a 22-gauge needles at about 2-mm away from the edge of the mesh, and BPUR meshes were sterilized by UV exposure in a culture hood for 1 h before use. PEGylated human fibrinogen (Sigma, St. Louis, Mo.) was prepared, for example, as described in O. M. Benavides, J. P. Quinn, S. Pok, J. Petsche Connell, R. Ruano, J. G. Jacot, Capillary-like network formation by human amniotic fluid-derived stem cells within fibrin/poly(ethylene glycol) hydrogels, Tissue Eng. Part A 21(7-8) (2015) 1185-94, which is hereby incorporated by reference herein in its entirety. For example, Human fibrinogen (F3879; Sigma-Aldrich, St Louis, Mo.) was solubilized in phosphate-buffered saline (PBS) at a concentration of 40 mg/ml. After 1 h of incubation at 37° C. and brief vortexing, the solution was sterilized using a 0.20-μm filter. Succinimidyl glutarate-modified bifunctional PEG (3.4 kDa SG-PEG-SG; NOF America Corporation, White Plains, N.Y.) was dissolved in PBS at 4 mg/ml and syringe filtered. Fibrinogen and PEG solutions were combined in a 1:1 volume ratio, mixed thoroughly, and incubated at 37° C. for 1 h.

Gel patches were fabricated in a sterile Teflon mold 7-mm in diameter and 2-mm deep. The PEG-fibrin gel was made with 30 μl sterile PBS containing 20 mg/ml of PEGylated human fibrinogen, adding 30 μl sterile ice-cold 40 mM CaCl₂ containing 20 U/ml human thrombin (Sigma, St. Louis, Mo.) and mixing well to promote gel formation. The storage and loss moduli of the PEGylated fibrin gels were measured using a parallel-plate rheometer (Discovery Hybrid 2; TA Instruments). PEG-Fibrin gels (80 μl) were formed directly between the plates at a gap of 1400 μm. Samples were allowed to gel for 5 minutes at 37° C. before being compressed 20% (gap of 1120 μm). Four replicates were subjected to shear at 1% strain through a dynamic angular frequency range of 0.1 to 100 rad/s. The elastic modulus was calculated from the linear region of the storage modulus using Hooke's law. Poisson's ratio was assumed to be 0.5, corresponding to an incompressible material (FIGS. 1F and G). For implantable patch fabrication, a BPUR mesh was laid on the top of fibrin gel immediately after gelation and another 10 μl of PEGylated fibrinogen solution was added on the top of the BPUR layer to enhance integration. Molds were incubated for 30 min at 37° C. and 5% CO₂ before patches were detached (FIG. 1E) and implanted onto heart defects.

For a control, bovine pericardium patches (7 mm in diameter, 270 μm in thickness) were cut from commercially available glutaraldehyde-fixed bovine pericardium (St. Jude Medical, Saint Paul, Minn.).

### Example 2—Patch Implantation

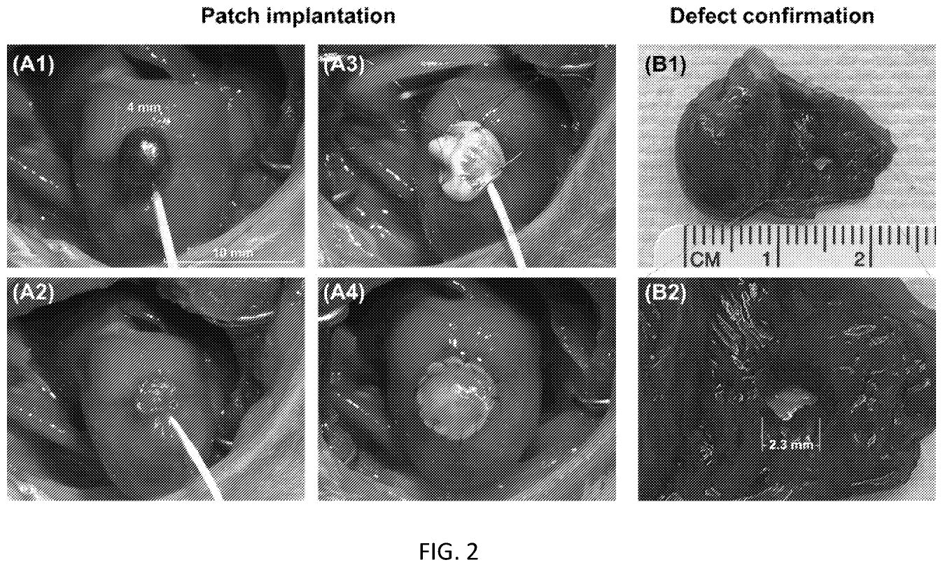

Experiments were performed to implant the cardiac patch, as described above in Example 1, over a mammalian heart defect. Sixty-two male Sprague-Dawley rats weighing 400 ±10 g (Envigo, Cambridgeshire, UK) were randomly assigned to a 4-week sham group (Sham, n=10), a 4-week glutaraldehyde-fixed bovine pericardium group (Pericardium, n=10), a 4-week BPUR-reinforced hydrogel patch group (BPUR PEG-fibrin, n=10), an 8-week sham group (n=11), an 8-week pericardium group (n=10), or an 8-week BPUR PEG-fibrin group (n=11). Rats were anesthetized using 5% isoflurane inhalation with 100% oxygen followed by intubation and respiratory support with a rodent mechanical ventilator (Harvard Apparatus, Holliston, Mass.) at a peak inspiratory pressure of 11 cmH₂O and 75 beats/minute. Surgery procedures were performed in a sterile environment on a controlled heating pad. An Animal Bio Amp (FE 136, ADInstruments) and an Animal Oximeter Pod (ML325, ADInstruments) that attached to a PowerLab 4/30 system (ADInstruments, Spring, Colo.) were used to monitor electrocardiogram and SpO₂. Anesthesia was maintained with 2% isoflurane inhalation with 100% oxygen. The rat heart was exposed via a 3-cm incision through a 4th left thoracotomy, for example, as described in Z. W. Tao, L. G. Li, Z. H. Geng, T. Dang, S. J. Zhu, Growth factors induce the improved cardiac remodeling in autologous mesenchymal stem cell-implanted failing rat hearts, J Zhejiang Univ Sci B 11(4) (2010) 238-48 (herein “Tao”), which is hereby incorporated by reference herein in its entirety. FIGS. 2A1-4 show an exemplary surgical procedure on a rat heart to create a defect and implant a patch of the present disclosure. For example, as shown in FIG. 2A1, a purse string suture, 4 mm in diameter, was created on the RV free wall with a 6-0 polypropylene suture (Ethicon, US). Both ends of the stitch suture were passed through a 22-gauge plastic vascular cannula (VWR International, Radnor, Pa.) that served as a tourniquet to secure the purse string. As shown in FIG. 2A2, the distend portion was excised to create a full-thickness defect. For example, as shown in FIGS. 2B1 and 2B2, three quarters of the bulging part of the purse string was excised to create approximately a 2-3 mm full-thickness defect contacting blood. As shown in FIG. 2A3, the patch was stitched over the defect with 7-0 polypropylene suture (Ethicon, US), first fixed by 4 stitches at positions of 90°, 180°, 270° and 360°, then continuously sutured fully around the patch. As shown in FIG. 2A4, the purse string suture was released. Animals in the sham group experienced the same chest opening and pericardium tearing, but no defect was created and no patch was sutured onto the RV free wall. The muscle layers of the chest and the skin were closed with a 4-0 polyglactin absorbable suture (AD Surgical, Sunnyvale, Calif., USA).

Isoflurane supply was stopped immediately after the skin layer was closed. Before animals regained consciousness, Meloxican (5 mg/ml, MWI Animal Health, Grand Prairie, Tex., USA) 0.5 mg/kg was administered subcutaneously once to reduce post surgery pain. One animal in the 4-week pericardium group and 1 animal in the 8-week BPUR PEG-fibrin group died from massive bleeding during surgery, and 1 animal in the 4-week BPUR PEG-fibrin group died from acute RV myocardial infarction during surgery.

### Example 3—Cardiac Function Assessment

Experiments were performed to assess cardiac function resulting from implantation of a cardiac patch of the present disclosure over a mammalian heart defect. As one example, to assess left ventricular (LV) function, echocardiography was performed at the end of the 8-week time point. Animals were anesthetized and placed on a controlled heating pad, and anesthesia was maintained with 2% isoflurane inhalation with 100% oxygen. Standard transthoracic echocardiography was performed using the GE Vivid 7 system (GE Vingmed Ultrasound AS, N-3190 Horten, Norway) fitted with an GE S10 transducer. LV parameters were obtained from two dimensional images and M-mode interrogation in parasternal short-axis and long-axis view as described in Tao, and then LV end-diastolic dimensions (LVEDd), ejection fraction (LVEF) and fractional shortening (LVFS), and end-diastolic area (LVEDA) were calculated. Echocardiographic measurements were averaged from at least five cardiac cycles.

Electrocardiogram (ECG) may also be used to assess cardiac function. For example, ECG signals were recorded at 4- and 8-week post-implantation endpoints. Animals were anesthetized using 5% isoflurane inhalation with 100% oxygen followed by intubation and respiratory support with a rodent mechanical ventilator, placed on a controlled heating pad, and then anesthesia was maintained with 2% isoflurane inhalation with 100% oxygen. An ECG signal was recorded for 30 min with an Animal Bio Amp that attached to a PowerLab 4/30 system, by inserting a needle anode (MLA1213, ADInstruments) into the left front leg of the animal, a needle cathode into the right front leg, and using the testis skin as ground. LabChart (ADInstruments) was used for analysis of malicious arrhythmia, categorized as frequent atrial premature beats (APBs), atrial tachycardia (AT), atrial fibrillation, frequent ventricular premature beats (VPBs), ventricular tachycardia, and ventricular fibrillation.

Cardiac catheterization may be performed to assess hemodynamics in hearts implanted with the disclosed device. For example, after recording ECG signals, the heart was exposed through a 5th left thoracotomy and a 2F micromanometer tipped catheter (SPR-869 Millar Instruments, Houston, Tex.) was inserted into the LV apex, and advanced into the LV to obtain LV pressure and conductance. After stabilization for 15 minutes, the signals were digitized at a sampling rate of 1 kHz/s using MPVS-300 (Millar Instruments) and were acquired to a PowerLab 4/30 system at steady state. LabChart Pro v.8.10 software with the pressure-volume (PV) loop module (ADInstruments) was utilized for subsequent assessment of LV hemodynamic parameters. Heart rate (HR), LV systolic pressure (LVSP), LV end-diastolic pressure, maximal slope of systolic pressure increment (LV dP/dtmax) and diastolic pressure decrement (LV dP/dtmin), ejection fraction (LVEF), stroke volume (SV), end-diastolic volume (EDV), cardiac output (CO), and stroke work (SW) were computed using the cardiac PV-loop module. After completion of the hemodynamic assessment from of the LV, the catheter was inserted into the RV apex and advanced into the RV to acquire RV hemodynamics including systolic pressure (RVSP), RV end-diastolic pressure (RVEDP), maximal slope of systolic pressure increment (RV dP/dtmax), and diastolic pressure decrement (RV dP/dtmin). A 20-gauge IV catheter (VWR International, Radnor, Pa.) was inserted into the right jugular vein. After a stable signal was recorded from either LV or RV, 20 μl hypertonic saline (30%) bolus injection at least 2-3 times were performed for both ventricles to obtain a value for Vp for the saline calibration. After hemodynamic measurements were made under anesthesia, animals were euthanized with cardiac arrest by apical injection of 1 ml of 10% KCl. Hearts were excised, weighed, placed in a peel-away disposable embedding mold (VWR International, Radnor, Pa.), frozen in liquid N2, and then immediately immersed in Tissue Tek OCT compound (VWR International, Radnor, Pa.) and placed in a −80° C. freezer.

In some embodiments, immunohistochemistry may be conducted and histology may be studied. As one example, heart samples were sliced using a cryostat (Cryotome E, Thermo Shandon). Whole heart longitudinal sections, directly through the middle of the defect, from the base to apex of the heart were cut at a thickness of 10 μm. The sections were placed on VWR Microslides for preparation of morphological and immunofluorescence examinations. For measurements of patch implantation-induced fibrosis and defect thickness, whole heart sections were stained with Masson's trichrome reagents (Sigma) according to the manufacturer's protocol. Section images (200× magnification) were taken under Zeiss 2.1 microscope (Germany), and the images of whole heart sections were stitched together using the Series feature within the Zeiss microscopy software. The whole scar area (mm2) and the patch material remaining area (mm2) were measured by tracing the edge of the scar and the edge of the remaining patch materials in each patch area. The patch implantation-induced fibrosis area was calculated as the whole defect area minus the patch material remaining area. The size of the external scar was measured and expressed as the external curve length (mm) in each sample and averaged; the size of the internal defect was measured between the internal muscle breaks and expressed as the internal curve length (mm) in each sample and averaged.

For immunofluorescence staining, whole heart sections of 10 μm thickness directly through the middle of the defect were fixed in 4% paraformaldehyde at 4° C. for 20 min; nonspecific epitope antigens were blocked with 10% goat serum (Sigma) at room temperature for 45 minutes. Sections were incubated with specific mouse anti-α-actinin antibody (1:200, Sigma, A7811), rabbit anti-cardiac troponin T (1:200, Invitrogen, MA5-12960), mouse anti-vimentin (1:200, Sigma, C9080), rabbit anti-von Willebrand factor (vWF; 1:750, Abcam, ab6994), mouse anti-α-smooth muscle actin (α-SMA; 1:200, Sigma, C6198), rabbit anti-CD45 (1:200, Abcam, ab10558), mouse anti-CD68 (1:200, Invitrogen, MA5-16654), rabbit anti-CD206 (1:200, Abcam, ab64693), Alexa Fluor 488 (1:40, Invitrogen, A12379) and 546 (1:40, Invitrogen, A22283) phalloidin at room temperature for 1 hour. Subsequently, sections were treated with goat anti-mouse or goat anti-rabbit secondary antibodies (1:400, Invitrogen, Alexa Fluor 488, Alexa Fluor 546, and Alexa Fluor 647) at room temperature for 1 hour. Nuclei were counterstained with 4,6-diamidino-2-phenylindole (DAPI; 2.5 μg/ml) for 5 minutes at room temperature. Fluorescent images were obtained with a Zeiss 2.1 microscope. For determination of granular-like tissue, the volume of vimentin positive signals (staining fibroblasts and endothelial cells) were measured by whole defect area×intensity mean value in the section. For evaluation of blood vessels, the total number of vWF positive signals was counted from 5 random 400×magnification patch material-centered ocular fields in the section. For evaluation of acute inflammation, the volume of CD45, CD68 and CD206 positive signals (staining leukocytes, pan-macrophages and M2 macrophages respectively) were measured with the average of 3 random 200×magnification patch material-centered ocular fields calculated by area×intensity mean value in each section.

### Example 4—Properties and Outcomes Resulting from Use of a Polyurethane-Reinforced Hydrogel Cardiac Patch

Experimental results were analyzed to determine outcomes resulting from the implantation of a cardiac patch of the present disclosure over a mammalian heart defect. Results are presented as mean±standard deviation. ECG arrhythmia was analyzed by Chi-square (and Fisher's exact) test. Comparisons between two groups were made using the independent-samples t-test, and comparisons among three groups were made using a one-way analysis of variance followed by a Tukey post hoc comparison test. Differences were considered statistically significant at a value of p<0.05.

FIGS. 1A-G show graphics, images, and a table illustrating various properties of a fabricated biodegradable poly(ether ester urethane) urea (BPUR)-reinforced hydrogel patch. For example, FIG. 1A shows a graph of a Fourier transform infrared (FTIR) spectrum of BPUR. For example, the peak at 1260 cm-1 corresponds to the ester group in PEG, while the peak at 1730 cm-1 corresponds to the carbonyl in urethanes and esters, and the peaks at 3333 and 1630 represent N-H stretching and carbonyls in ureas, respectively. The absence of a peak at 2267 cm-1 suggests negligible unreacted NCO. FIG. 1B shows a table of peak assignments of BPUR. FIG. 1C shows an image of a BPUR reinforcement layer (7 mm in diameter, 80 μm in thickness) cut from an electrospun mesh. FIG. 1D shows a scanning electron microscopy image of an electrospun BPUR. FIG. 1E shows a BPUR-reinforced fibrin gel patch (7 mm in diameter, 1 mm in thickness). FIG. 1F shows a graph of Young's modulus (893±193 Pa). FIG. 1G shows a graph depicting a frequency spectrum of the fibrin gel (n=4).

In some embodiments, survival rates may be determined. For example, in the study discussed above, one animal in the 8-week pericardium group died from cardiac arrest the second day post surgery, and 1 animal in the 8-week BPUR PEG-fibrin group became paraplegic and was euthanatized the second day post surgery. All other animals surviving the surgery survived to the endpoint (57/62 total rats).

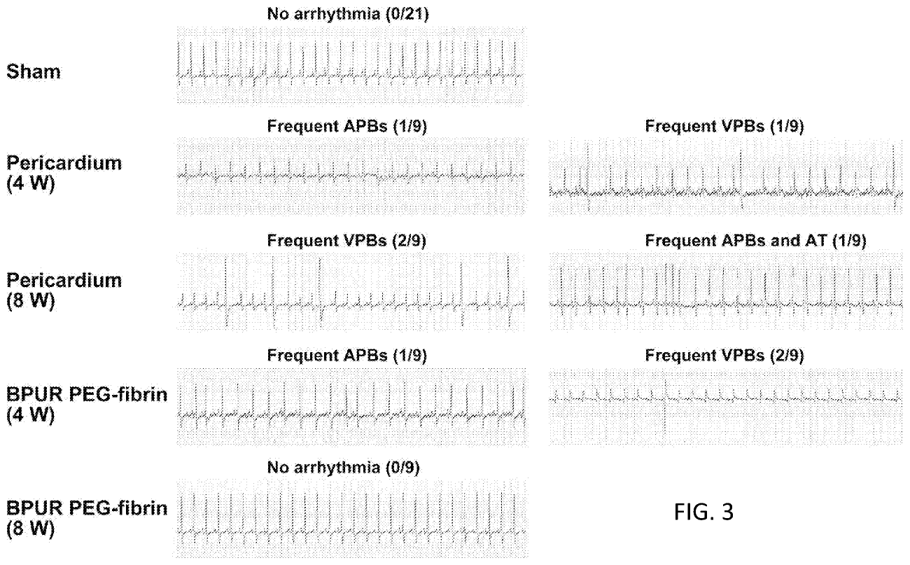

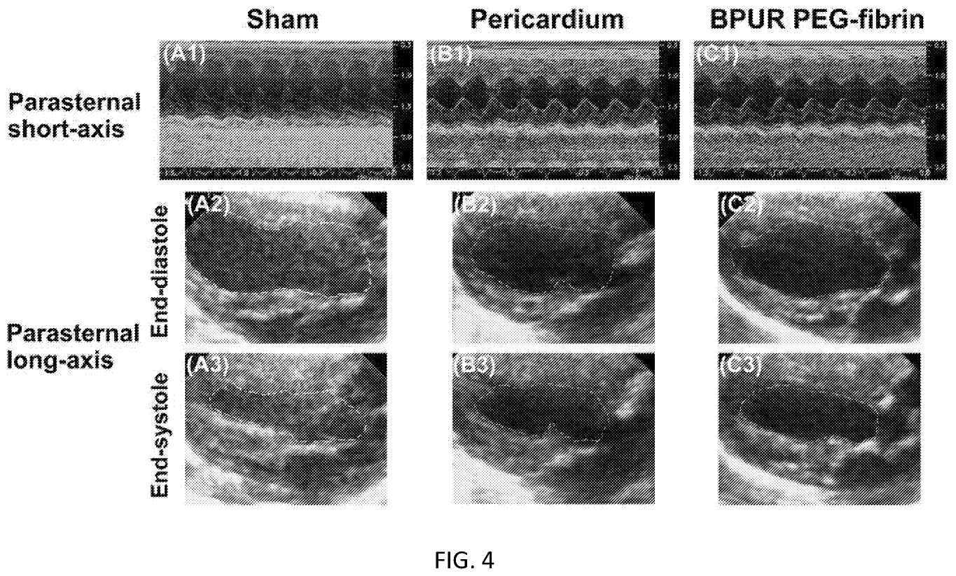

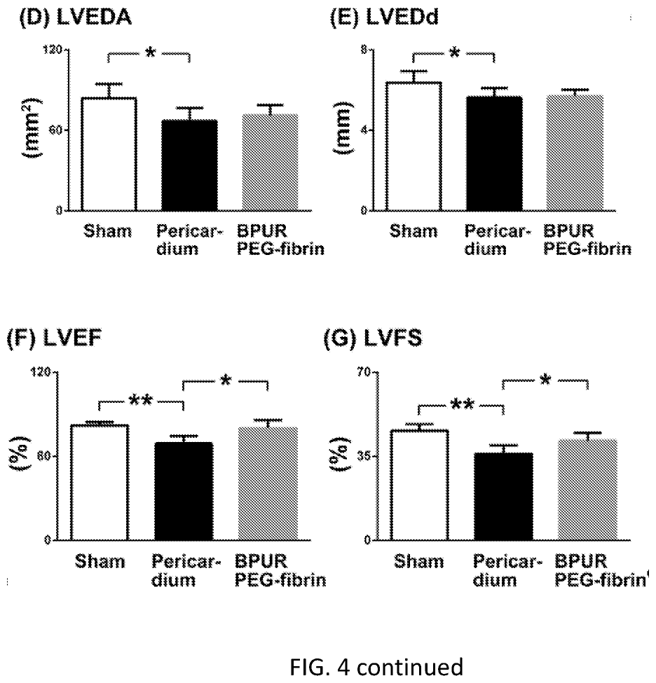

ECG was also used to assess arrhythmia in the test subjects. These results are depicted at FIG. 3, which shows ECG arrhythmia recorded for 30 min at 4- and 8-weeks post surgery for the study discussed above. As shown, no animals with sham surgery had arrhythmia at either endpoint. At the 4-week endpoint, 2 animals (2/9) in the pericardium group had arrhythmia, 1 with frequent atrial premature beats (APBs) and 1 with frequent ventricular premature beats (VPBs); 3 animals (3/9) in the BPUR PEG-fibrin group had arrhythmia, 1 with frequent APBs and 2 with frequent VPBs. Fisher's exact test showed no significant difference (p=1.000). At the 8-week endpoint, 3 animals (3/9) in the pericardium group had arrhythmia, 2 with frequent VPBs and 1 with frequent APBs plus atrial tachycardia (AT); no arrhythmia was found in any rats in the BPUR PEG-fibrin group. Fisher's exact test showed no significant difference (p=0.2059). Echocardiography was also used to assess heart function on animals at 8 weeks post surgery. FIGS. 4A-G show images and graphs depicting changes in the function of a left ventricle (LV) at 8 weeks post surgery. FIGS. 4A1, B1, and C1 show M-mode images from the parasternal short-axis view. FIGS. 4A2, B2, and C2 show B-mode images from the parasternal long-axis view indicating the LV end-diastolic area (LVEDA). FIGS. 4A3, B3, and C3 show B-mode images from the parasternal long-axis view indicating the LV end-systolic area. FIGS. 4D-G show graphs depicting changes in the LV end-diastolic area (LVEDA), LV end-diastolic dimensions/diameter (LVEDd), LV ejection fraction (LVEF), and LV fractional shortening (LVFS) post surgery. Values are mean ±standard deviation. The single asterisk (*) represents p<0.05, while the double asterisk (**) represents p<0.01.

As shown, implantation of a fixed pericardium patch at 8 weeks post surgery resulted in a significant decrease in LVEDA (67.0±9.7 mm2, n=6; FIG. 4D) and LVEDd (5.63±0.46 mm, n=6; FIG. 4E) when compared with LVEDA (83.8±10.6 mm2, n=6, p<0.05) and LVEDd (6.37±0.56 mm, n=6, p<0.05) in the sham group; implantation of BPUR PEG-fibrin patch slightly decreased LVEDA (71.0±7.7 mm2, n=6) and LVEDd (5.70±0.32 mm, n=6), but there was no significant difference when compared with either the sham group or the fixed pericardium group. Implantation of a fixed pericardium patch at 8 weeks post surgery significantly decreased LVEF (69.2±5.3%, n=6; FIG. 4F) and LVFS (36.0±3.5%, n=6; FIG. 4G) when compared with LVEF (82.0 2.3%, n=6) and LVFS (45.7±2.7%, n=6) in the sham group (p<0.01); however, the LVEF (80.2 ±5.7%, n=6) and LVFS (41.5±3.3%) were significantly larger (p<0.05) in the BPUR PEG-fibrin patch group compared with the pericardium group.

Hemodynamics were also assessed. For example, Table 1 depicted below shows resulting body weight (BW), heart weight (HW), HW/BW, and heart rate (HR), as well as other ventricular hemodynamics measurements at 4- and 8-weeks post surgery. For example, the table below further shows right ventricular systolic pressure (RVSP); right ventricular end-diastolic pressure (RVEDP); right ventricular maximal slope of pressure increase (RV dP/dtmax); right ventricular maximal slope of pressure decrease (RV dP/dtmin); right ventricular end-diastolic volume (RVEDV); right ventricular stroke volume (RV SV); right ventricular ejection fraction (RVEF); right ventricular stroke work (RV SW); right ventricular cardiac output (RV CO); left ventricular systolic pressure (LVSP); left ventricular end-diastolic pressure (LVEDP); left ventricular maximal slope of pressure increase (LV dP/dtmax); left ventricular maximal slope of pressure decrease (LV dP/dtmin); left ventricular end-diastolic volume (LVEDV); left ventricular stroke volume (LV SV); left ventricular ejection fraction (LVEF); left ventricular stroke work (LV SW); and left ventricular cardiac output (LV CO). Values are mean±standard deviation. The single asterisk (*) represents p<0.05, and the double asterisk (**) represents p<0.01 vs Sham; while the single hashtag (#) represents p<0.05, and the double hashtag (##) represents p<0.01 vs Pericardium.

TABLE 1
Body weight, heart weight, and ventricular hemodynamics measurements at 4-and 8-weeks post surgery.

| | 4 weeks | | | 8 weeks | | |
|---|---|---|---|---|---|---|
| | | | BPUR | | | BPUR |
| | Sham | Pericardium | PEG-fibrin | Sham | Pericardium | PEG-fibrin |
| | (n = 7) | (n = 8) | (n = 8) | (n = 6) | (n = 7) | (n = 7) |
| BW (g) | 469 ± 24 | 463 ± 11 | 461 ± 12 | 510 ± 24 | 483 ± 18* | 482 ± 9* |
| HW (g) | 1.27 ± 0.09 | 1.34 ± 0.13 | 1.28 ± 0.09 | 1.29 ± 0.05 | 1.36 ± 0.07 | 1.36 ± 0.09 |
| HW/BW (mg/g) | 2.70 ± 0.17 | 2.89 ± 0.23 | 2.75 ± 0.02 | 2.55 ± 0.17 | 2.81 ± 0.07* | 2.82 ± 0.21* |
| HR (bpm) | 311 ± 47 | 289 ± 30 | 295 ± 32 | 314 ± 29 | 296 ± 30 | 329 ± 34 |
| | (n = 7) | (n = 8) | (n = 8) | (n = 6) | (n = 7) | (n = 7) |
| RVSP (mmHg) | 29.9 ± 2.7 | 26.2 ± 2.7* | 26.1 ± 2.3* | 30.8 ± 3.9 | 25.7 ± 3.4* | 29.7 ± 3.9 |
| RVEDP (mmHg) | 3.6 ± 2.2 | 4.9 ± 2.2 | 3.5 ± 2.4 | 4.7 ± 1.9 | 5.0 ± 2.4 | 4.6 ± 1.7 |
| RV dP/dtₘₐₓ | 1241 ± 165 | 1190 ± 189 | 1201 ± 173 | 1397 ± 168 | 1112 ± 185** | 1373 ± 64^{#} |
| (mmHg/s) | | | | | | |
| RV dP/dtₘᵢₙ | 986 ± 251 | 851 ± 160 | 907 ± 106 | 1173 ± 178 | 779 ± 194** | 1139 ± 191^{##} |
| (mmHg/s) | | | | | | |
| | (n = 6) | (n = 5) | (n = 6) | (n = 6) | (n = 6) | (n = 6) |
| RVEDV (μl) | 181.7 ± 19.5 | 150.8 ± 12.0* | 155.3 ± 13.1* | 187.3 ± 11.8 | 164.8 ± 11.4** | 169.3 ± 9.9* |
| RV SV (μl) | 133.4 ± 21.1 | 81.8 ± 9.1** | 95.9 ± 9.0** | 130.6 ± 4.8 | 92.9 ± 8.7** | 109.4 ± 6.2**^{#} |
| RVEF (%) | 73.6 ± 7.5 | 54.3 ± 6.3* | 62.0 ± 7.5* | 72.2 ± 5.7 | 56.4 ± 4.8** | 65.0 ± 4.6^{#} |
| RV SW | 2.56 ± 0.24 | 2.08 ± 0.16* | 2.34 ± 0.12 | 2.58 ± 0.25 | 2.17 ± 0.18** | 2.41 ± 0.19 |
| (mmHg · ml) | | | | | | |
| RV CO (ml/min) | 27.5 ± 2.5 | 23.1 ± 2.8* | 26.4 ± 1.8 | 27.4 ± 1.2 | 23.0 ± 2.1** | 26.2 ± 1.9^{#} |
| | (n = 7) | (n = 8) | (n = 8) | (n = 6) | (n = 7) | (n = 7) |
| LVSP (mmHg) | 116.3 ± 8.4 | 100.7 ± 7.7** | 105.2 ± 8.4* | 127.7 ± 13.8 | 107.5 ± 9.4* | 114.7 ± 10.3 |
| LVEDP (mmHg) | 5.8 ± 1.6 | 5.4 ± 2.8 | 6.2 ± 1.6 | 6.5 ± 1.3 | 7.2 ± 1.1 | 6.3 ± 1.3 |
| LV dP/dtₘₐₓ | 5954 ± 872 | 5951 ± 733 | 5932 ± 1177 | 7046 ± 1197 | 5838 ± 738 | 6448 ± 834 |
| (mmHg/s) | | | | | | |
| LV dP/dtₘᵢₙ | 5223 ± 808 | 5325 ± 910 | 5292 ± 1258 | 6236 ± 957 | 4981 ± 847 | 5728 ± 1217 |
| (mmHg/s) | | | | | | |
| LVEDV (μl) | 208.0 ± 28.8 | 169.4 ± 30.2* | 190.6 ± 17.6 | 196.8 ± 19.8 | 169.6 ± 12.8* | 175.2 ± 16.8 |
| LV SV (μl) | 137.6 ± 23.0 | 86.8 ± 14.2** | 105.8 ± 18.2* | 128.8 ± 12.4 | 103.4 ± 12.8** | 120.8 ± 9.6^{#} |
| LVEF (%) | 69.6 ± 4.7 | 49.8 ± 5.8** | 55.1 ± 5.2** | 70.2 ± 6.3 | 54.5 ± 3.5** | 63.1 ± 4.9*^{#} |
| LV SW | 14.9 ± 3.8 | 8.9 ± 2.7** | 12.7 ± 2.4^{#} | 15.1 ± 2.4 | 10.0 ± 3.8** | 14.2 ± 1.8^{#} |
| (mmHg · ml) | | | | | | |
| LV CO (ml/min) | 43.7 ± 8.1 | 24.8 ± 6.7** | 34.9 ± 5.5^{#} | 38.0 ± 4.9 | 30.2 ± 4.3* | 37.5 ± 4.3^{#} |

As shown in Table 1, four weeks after surgery, BW, HW, HW/BW, and HR were not significantly different between any experimental groups. Eight weeks after surgery, BW was significantly decreased (p<0.05) and HW/BW ratio was significantly increased (p<0.05) in both the Pericardium and the BPUR PEG-fibrin groups compared with the Sham group. As shown, LVSP, LVEDV, LVEF, LV SW and LV CO at both 4- and 8-weeks were dramatically decreased in the pericardium group, and LVSP and LVEF at 4 weeks, and LVEF at 8 weeks were dramatically decreased in the BPUR PEG-fibrin group compared with the sham control (p<0.05; p<0.01). LVEF was higher at 8 weeks, and LV SW and LV CO were higher at both 4-and 8-weeks post surgery in the BPUR PEG-fibrin group compared with the pericardium group (p<0.05).

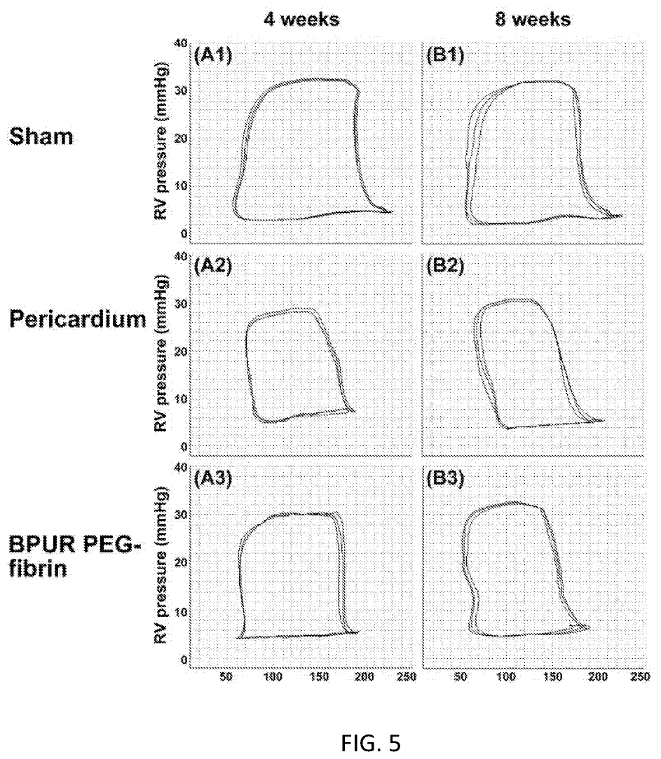

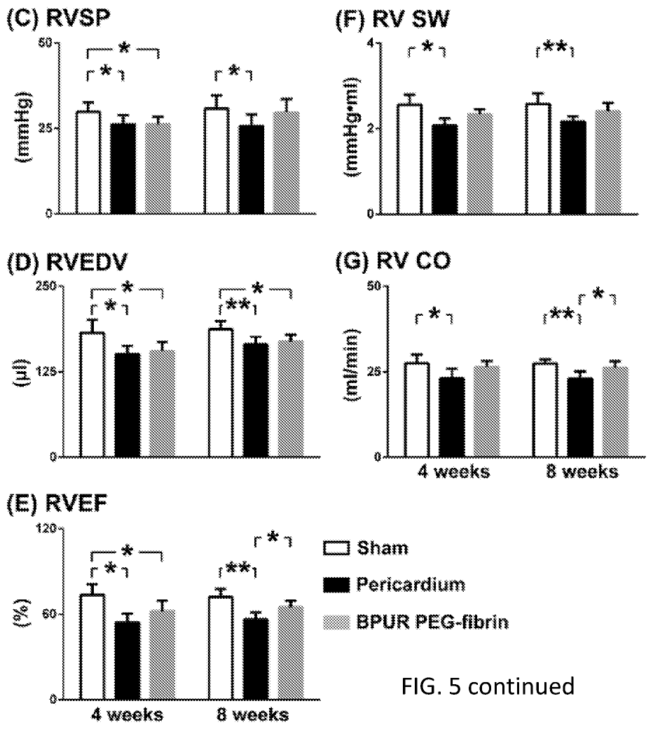

RV pressure-volume (PV) may also be measured to assess heart function. For example, FIG. 5 shows graphs depicting changes of an RV PV loop at 4- and 8-weeks post surgery. FIGS. 5A1-3 show the RV PV loop at 4 weeks post surgery. FIGS. 561-3 show the RV PV loop at 8 weeks post surgery. FIGS. 5C-G show RV hemodynamics. For example, FIG. 5C shows right ventricular systolic pressure (RVSP); FIG. 5D shows right ventricular end-diastolic volume (RVEDV); FIG. 5E shows right ventricular ejection fraction (RVEF); FIG. 5F shows right ventricular stroke work (RV SW); and FIG. 5G shows right ventricular cardiac output (RV CO) post surgery. Values are mean ±standard deviation. The single asterisk (*) represents p<0.05, while the double asterisk (**) represents p<0.01.

As shown in FIG. 5, at both 4 weeks (FIGS. 5A2 and A3) and 8 weeks (FIGS. 5B2 and B3), the RV PV loop was shifted to the left compared with the sham group (FIG. 5A1 and 61, respectively). RVSP, RVEDV and RVEF were significantly lower in the pericardium group at 4- and 8-weeks, and RVSP, RVEDV and RVEF were significantly lower in the BPUR PEG-fibrin group at 4 weeks and RVEDV was significantly lower at 8 weeks post surgery compared with the sham group (p<0.05; p<0.01). However, RVEF was significantly higher in the BPUR PEG-fibrin group at 8 weeks post surgery compared with the pericardium group (p<0.05). RV SW and RV CO were both significantly lower in the pericardium group at both 4- and 8-weeks post surgery compared with the sham group (p<0.05; p<0.01); however, RV CO was significantly higher in the BPUR PEG-fibrin group at 8 weeks post surgery (p<0.05) compared with the pericardium group. At 8 weeks post surgery, RV dP/dtmax and RV dP/dtmin were significantly lower in the pericardium group compared with the sham control (P<0.01); in contrast, RV dP/dtmax and RV dP/dtmin in the BPUR PEG-fibrin group at 8 weeks post surgery were not significantly different from the sham group, but were significantly higher compared with the pericardium group (p<0.05; p<0.01) (e.g., see Table 1).

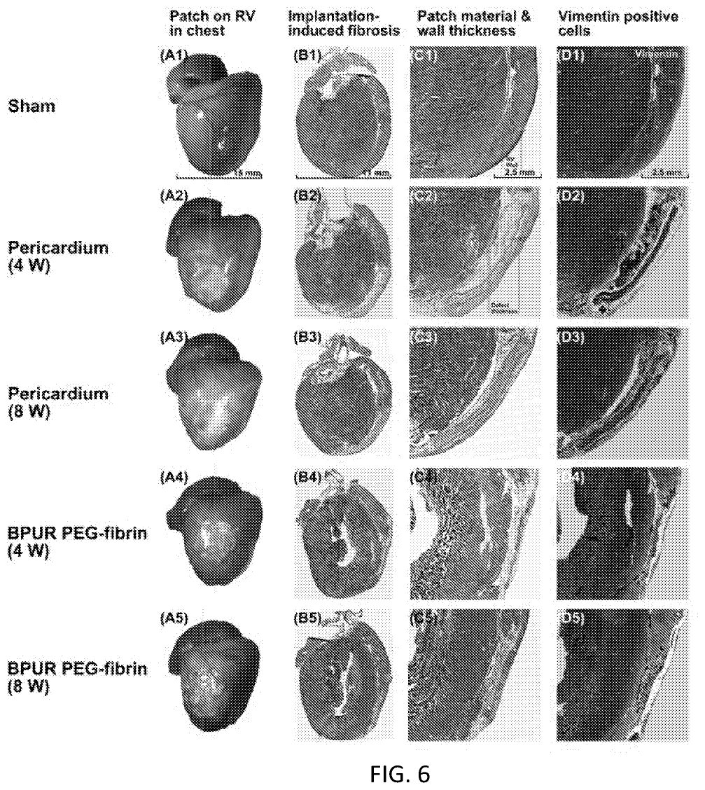

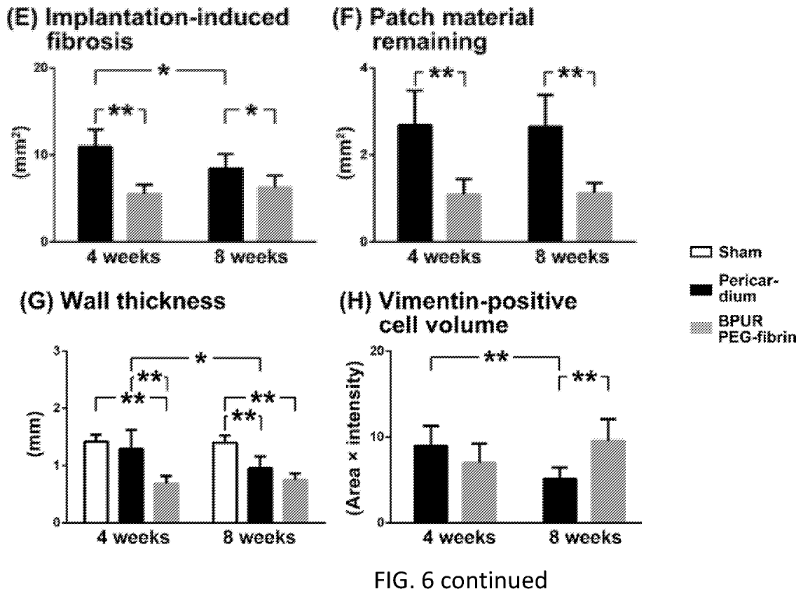

Histology may be performed to assess levels of fibrosis, infiltration, degredation, etc. in the inplanted heart patches. For example, FIGS. 6A-H show images and graphs depicting implantation-induced fibrosis, patch degradation, wall thickness and vimentin-positive cell volume at 4- and 8-weeks post surgery. FIGS. 6A1-A5 show macroscopic images of the patch area on the RV wall at 4- and 8-weeks post surgery. FIGS. 6B1-B5 show images of Masson's trichrome staining depicting patch implantation-induced fibrosis on sections directly through the center of the defect. FIGS. 6C1-C5 show images of insets depicting the degradation of patch materials and defect thickness. FIGS. 6D1-D5 show images depicting immunofluorescence staining showing filament actin (F-actin positive), cardiac fibroblasts and endothelial cells (vimentin positive), and nuclei (DAPI). FIGS. 6E-H show graphs depicting changes of patch implantation-induced fibrosis, patch material remaining, wall thickness and vimentin-positive cell volume. Values are mean ±standard deviation. A single asterisk (*) represents p<0.05, while a double asterisk (**) represents p<0.01.

At 4- and 8-weeks post surgery, one third of patch-implanted hearts exhibited minimal thoracic adhesions. Neither group showed any dehiscence or aneurysm formation at the site of the implanted patch. In the BPUR PEG-fibrin group, the BPUR support layer degraded to an apparent loss of structure integrity and was replaced with native-like tissue at 4 weeks (FIG. 6A4) and further at 8 weeks (FIG. 6A5); however, the pericardium group showed no degradation or native-like tissue replacement at both 4 weeks (FIG. 6A2) and 8 weeks (FIG. 6A3) post surgery.

Masson's trichrome staining was used to evaluate fibrosis and wall thickness. As shown in FIGS. 6B1-B5, the pericardium group had significantly higher fibrotic area at both 4- and 8-weeks (10.93±1.97 mm², n=7 and 8.43±1.67 mm², n=7) compared with the BPUR PEG-fibrin group (5.52±1.06 mm², n=7 and 6.23±1.39 mm², n=7) (p<0.01; p<0.05), but the fibrotic area was significantly smaller at 8 weeks compared with at 4 weeks post surgery in the pericardium group (p<0.05) (FIG. 6E).

Areas of remaining patch material were measured to quantify degradation. s shown in FIGS. 6C1-C5, the patch material area in the pericardium group at 4 weeks (2.68 ±0.80 mm², n=7) and 8 weeks (2.65±0.73 mm², n=7) was greater than in the BPUR PEG-fibrin group (1.09 ±0.35 mm², n=7 and 1.12±0.23 mm², n=7) (p<0.01) (FIG. 6F). The wall thickness in the pericardium group at 4 weeks (1.29±0.33 mm, n=7) was greater than the BPUR PEG-fibrin group (0.69±0.13 mm, n=7) (p<0.01). The wall thickness in the pericardium group at 8 weeks (0.95±0.21 mm, n=7) was smaller than at 4 weeks (p<0.05). There was no significant difference between wall thicknesses in the pericardium group and the BPUR PEG-fibrin group (0.75±0.11 mm, n=7) at 8 weeks post surgery. Compared with the normal thickness of the RV wall at 4 weeks (1.42±0.12 mm, n=6) and 8 weeks (1.43±0.11 mm, n=6), the wall thickness in the BPUR PEG-fibrin group at 4- and 8-weeks and the wall thicknesses in the pericardium group at 8 weeks were much smaller (p<0.05; p<0.01) (FIG. 6G).

Vimentin expression and vascularization was determined to assess tissue repair. For example, immunofluorescence staining of vimentin in the center of the patched area at 4- and 8-weeks post surgery was qualified and used as a measure of granulation tissue (FIG. 6D1-5). There were fewer vimentin-positive cells in the patch area in the pericardium group (FIG. 6D2 and D3) compared with the BPUR PEG-fibrin group (FIG. 6D4 and D5), indicating less granulation tissue (e.g., tissue repair) with the pericardium group than with the BPUR PEG-fibrin group. As shown in FIG. 6H, when compared with vimentin expression volume at 8 weeks, there was a significant difference between the pericardium group (5.13±1.29, n=6) and the BPUR PEG-fibrin group (9.58±2.49, n=6) (p<0.01). Further, the volume of vimentin-positive cells in the pericardium group at 8 weeks was significantly lower compared with 4 weeks (8.98±2.31, n=7) (p<0.01).

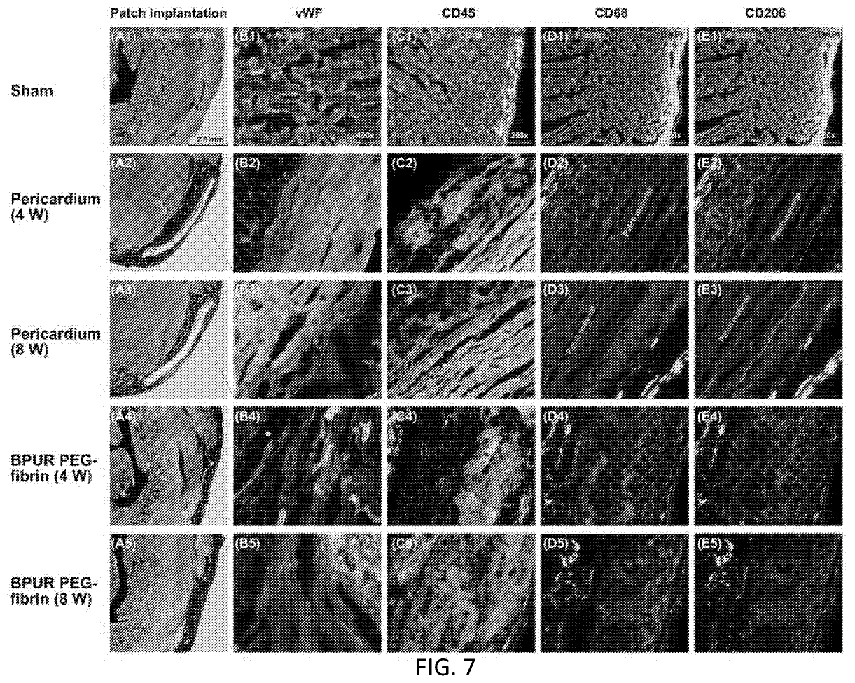

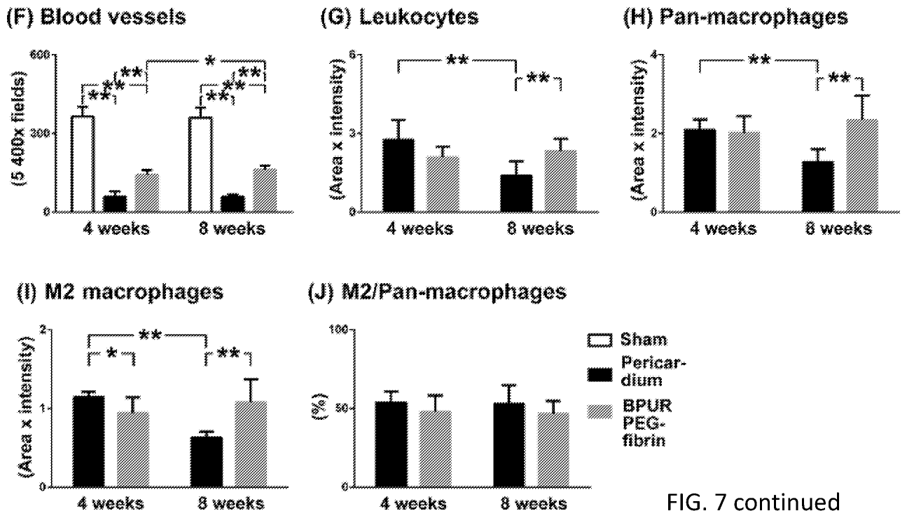

Vascularization and macrophage infiltration was measured, as shown in FIGS. 7A-J. Specifically, levels of vascularization and macrophage infiltration were measured at 4- and 8-weeks post surgery. FIGS. 7A1-A5 show images (200× magnification images stitched together) of an explanted patch stained for α-actinin, αSMA, vWF and DAPI. FIGS. 7B1-B5 show images of vWF staining indicating blood vessels. FIGS. 7C1-C5 show images of CD45 staining indicating leukocytes. FIGS. 7D1-D5 show images of CD68 staining indicating pan-macrophages. Figs. 7E1-E5 show images of CD206 staining indicating M2 macrophages. FIG. 7F shows a graph depicting the number of blood vessels. FIG. 7G shows a graph depicting the amount of CD45 expression indicating leukocytes. FIG. 7H shows a graph depicting the amount of CD68 expression indicating pan-macrophages. FIG. 71 shows a graph indicating the amount of CD208 expression indicating M2 macrophages. FIG. 7J shows a graph indicating a percentage of M2 over Pan-macrophages. Values are mean ±standard deviation. A single asterisk (*) represents p<0.05, while a double asterisk (**) represents p<0.01

To measure vascularization, the number of blood vessels may be counted using immunofluorescence staining. For example, the number of blood vessels was counted using the immunofluorescence staining of vWF in the patch material-centered area at 4- and 8-weeks post surgery (see, e.g., FIGS. 7B1-B5). As shown, there was little ingrowth of blood vessels in the pericardium group at 4 weeks (FIG. 7B2) and 8 weeks (FIG. 7B3) compared with the BPUR PEG-fibrin group at 4 weeks (FIG. 7B4) and 8 weeks (FIG. 7B5). The number of blood vessels in the BPUR PEG-fibrin group at 4 weeks (142.4±17.6, n=7) and 8 weeks (162.1 ±14.9, n=7) was greater than in the pericardium group at 4 weeks (59.4±19.9, n=7) and 8 weeks (59.0±8.2, n=7) post surgery (p<0.01); the number of blood vessels in the BPUR PEG-fibrin group at 8 weeks was greater than at 4 weeks (p<0.05). However, the number of blood vessels in the sham group at 4 weeks (365.0±36.1, n=7) and 8 weeks (360.5±38.2, n=7) was much greater than in the two patch implantation groups (p<0.01) (FIG. 7F).

Macrophage infiltration may also be measured with immunofluorescence staining. For example, immunofluorescence staining of CD45, CD68 and CD206 was used to evaluate the infiltration of leukocytes, especially neutrophils and monocytes, pan-macrophages, and M2 macrophages (FIGS. 7C1-C5, D1-5 and E1-5) respectively at 4- and 8-weeks post surgery. There was a significant difference in CD45 expression between the pericardium group (1.40±0.54, n=7) and the BPUR PEG-fibrin group (2.34±0.47, n=7) at 8 weeks (p<0.01), and the CD45 expression significantly decreased between 4 weeks (2.77±0.75, n=7) and 8 weeks in the pericardium group (p<0.01) (FIG. 7G). There was a significant difference in CD68 expression between the pericardium group (1.28±0.32, n=7) and the BPUR PEG-fibrin group (2.35±0.61, n=7) at 8 weeks (p<0.01), and the CD68 expression significantly decreased between 4 weeks (2.10±0.26, n=7) and 8 weeks in the pericardium group (P<0.01) (FIG. 6H). The CD206 expression was greater in the pericardium group (1.15±0.06, n=7) than in the BPUR PEG-fibrin group (0.94±0.02, n=7) at 4 weeks (p<0.05); but at 8 weeks, the CD206 expression was greater in the BPUR PEG-fibrin group (1.08±0.29, n=7) than in the pericardium group (0.63±0.08, n=7) (p<0.01); furthermore, the CD206 expression in the pericardium group significantly decreased between 4- and 8-weeks (p<0.01) (FIG. 61). The percentage of CD206/CD68 was also calculated, and no significant difference was found between the pericardium group and the BPUR PEG-fibrin group at either 4 weeks (53.9±6.9%, n=7 vs 47.8±10.5%, n=7) or 8 weeks (53.1±11.6%, n=7 vs 46.5±8.3%, n=7) (p>0.05) post surgery (FIG. 7J).

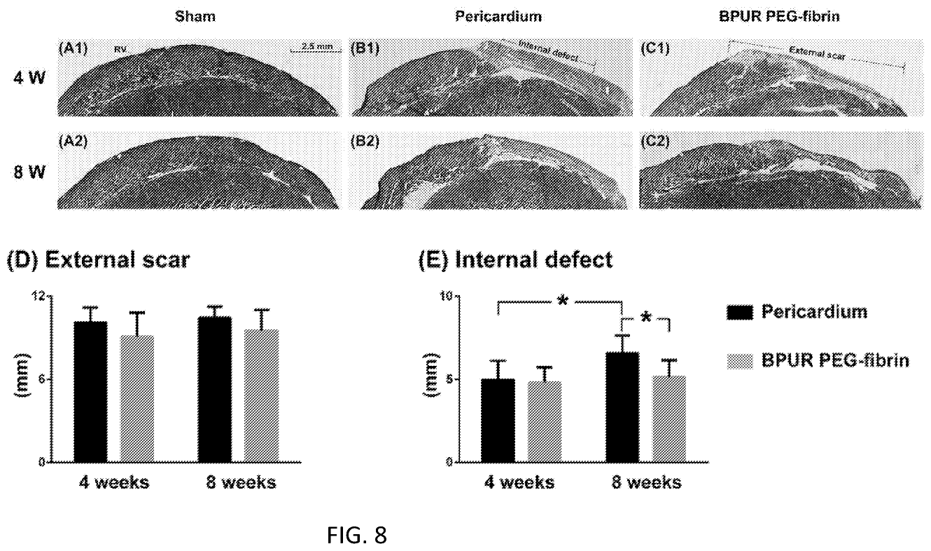

In several embodiments, the external scar size and the internal defect size may be measured. For example, FIGS. 8A-E show images and graphs depicting the external scar size and the internal defect size at 4- and 8-weeks post surgery. Values are mean±standard deviation. A single asterisk (*) represents p<0.05, while a double asterisk (**) represents p<0.01. As shown, the curve length of the external scar was not significantly different between the pericardium and the BPUR PEG-fibrin groups at 4 weeks (10.09±1.09 mm, n=7 vs 9.09±1.73 mm, n=7) and 8 weeks (10.45±0.79 mm, n=7 vs 9.51±1.49 mm, n=7); however, the curve length of the external scar was slightly higher in the two patch implantation groups at 4 weeks compared with at 8 weeks (p>0.05) (See, e.g., FIGS. 8B1-B2 and D). At 4 weeks, there was not a significant difference in the curve length of the internal defect between the pericardium group (4.97±1.14 mm, n=7) and the BPUR PEG-fibrin group (4.82±0.91 mm, n=7). At 8 weeks, the internal defect in the pericardium group grew dramatically larger (6.56±1.08 mm, n=7) compared with that at 4 weeks (p<0.05) and was also much larger than the BPUR PEG-fibrin group (5.14±1.01 mm, n=7) (p<0.05).

### Example 5—Conclusion

The above examples demonstrate that a cardiac patch comprised of PEG-fibrin reinforced by a BPUR mesh induced greater muscular and vascular ingrowth with a limited foreign body response compared to a commercial glutaraldehyde-crosslinked pericardium patch, resulting in improved heart function in an adult rat RV wall replacement model. At 8 weeks post surgery, rat hearts patched with BPUR PEG-fibrin had less fibrosis, a decreased patch material size, and increased infiltration of endothelial cells, leukocytes, pan-macrophages, and M2 macrophages compared to hearts patched with fixed pericardium. These regeneratively remodeled patches at 8 weeks resulted in fewer incidences of arrhythmia, greater RV function as shown by RVEF and RV CO, and greater LV function as shown by LVEF, LVFS, LV SW and LV CO compared to fixed pericardium. However, all patched hearts exhibited arrhythmias, decreased RV and LV function, and enlarged defect sizes compared with sham controls.

This study additionally found that the multilayer cardiac patch provided mechanical support of the full thickness defect and continued to support the ventricular wall as the material degraded and invading cells and secreted extracellular matrix replaced the implanted materials, as evidenced by the lack of dehiscence or aneurysm formation at the site of the implanted patch at both 4- and 8-weeks post surgery. The mechanical strength of the patch was provided by the electrospun BPUR mesh that had an average tensile modulus of 2.9±0.4 MPa, similar to the elastic modulus of a native muscle (approximately 10 MPa). Furthermore, this study showed that both the formation of granular tissue, indicated by vimentin-positive cell staining, and the infiltration of neutrophils, monocytes, and M2 macrophages were higher in rat hearts with BPUR-reinforced hydrogel patches than with pericardium patches at 8 weeks post surgery. Tissue regeneration and repair proceed in a cascade fashion beginning with a coagulation and inflammatory phase, followed by granulation tissue formation, which is characterized by proliferation of fibroblasts and new thin-walled, delicate capillaries, as well as infiltrated inflammatory cells in a loose extracellular matrix. Within the first few days after scaffold implantation, disruption of the tissue structure and subsequent cell damage initiates an acute inflammatory response with a rapid influx of innate immune cells, predominantly neutrophils, mast cells, and monocytes. Neutrophils and monocytes are of hematopoietic origin and are involved in phagocytosis and pathogen clearance. Upon activation, resident tissue macrophages are supplemented by an active recruitment of blood monocytes, which then differentiate into macrophages and dendritic cells in the scaffold. Depending on the scaffold properties, this is followed by an M1/TH1 cell dominated pro-inflammatory response or an M2/TH2 cell dominated pro-regenerative response. The former is characterized by the prolonged presence of M1 macrophages, and recruited fibroblasts typically acquire an activated phenotype, producing fibrous scar tissue. In contrast, the pro-regenerative process is dominated by M2 macrophages under influence of TH2 cell secreted cytokines.

This study also found that the BPUR-reinforced patch was more rapidly resorbed than the glutaraldehyde-fixed pericardium patch. This could be because M2 macrophages, which mediate regenerative remodeling, were more populous in the BPUR PEG-fibrin group than the pericardium group at 8 weeks. Additionally, faster degradation and more M2 macrophages coincided with a smaller defect size at 8 weeks post surgery in the BPUR PEG-fibrin group compared with the pericardium group. A regenerative remodeling response to the BPUR-reinforced hydrogel patch likely paved the way for better action potential conduction, resulting in the absence of arrythmia at 8 weeks post surgery, and improved mechanical performance in the patched area.

The defect sizes in this study, confirmed from images at postmortem, are about 2-3 mm in diameter; however, defects will be bigger in a heart under pressure and beating. In both patch materials, the defects grew larger both between 4- and 8-weeks post surgery, and wall thickness were thinner than the normal RV wall in the sham group, especially at 8 weeks post surgery.

### Example 6—Heart Patch Fabrication of Electrospun Scaffold Incorporating Polyethylene Oxide

Experiments were performed to create a cardiac patch with increased porosity, pore stability, cell infiltration. For example, sacrificial polyethylene oxide (PEO) particles were embedded within the electrospun scaffolds to increase both their pore size and porosity. As discussed above, polyurethane (PU) was first selected for electrospinning for its elastic behavior and strength, which simulate native cardiac tissue mechanics. However, when PU was electrospun with PEO, the PU was stretched and removal of the PEO particles resulted in pore collapse. To increase retention of pore size and shape, polycaprolactone (PCL) was incorporated into the PU material forming the scaffolds. PCL acts a stiffer material, and has been shown to be compatible with PEO,

Five different scaffold materials were made and tested. The materials comprised 25% gelatin mixed with 0% (magenta bars), 10% (orange bars), 20% (lime bars), 30% (green bars), or 75% (blue bars) biodegradable PU (e.g., synthesized according to Guan et al). The remaining portion of the material was PCL (80 kDa). These five composites were electrospun in the presence and absence of PEO (MW 8000) using a custom rotating co-electrospinning apparatus. Fiber size and pore size were measured with DiameterJ analysis of Scanning Electron Microscopy (SEM) images. A dynamic mechanical analyzer was used to measure tensile strength of the resulting scaffolds. Porosity was measured with the gravimetric method. Cell infiltration depth was assessed via H&E-stained slides of scaffolds seeded with human dermal fibroblasts (hDF) for two weeks.

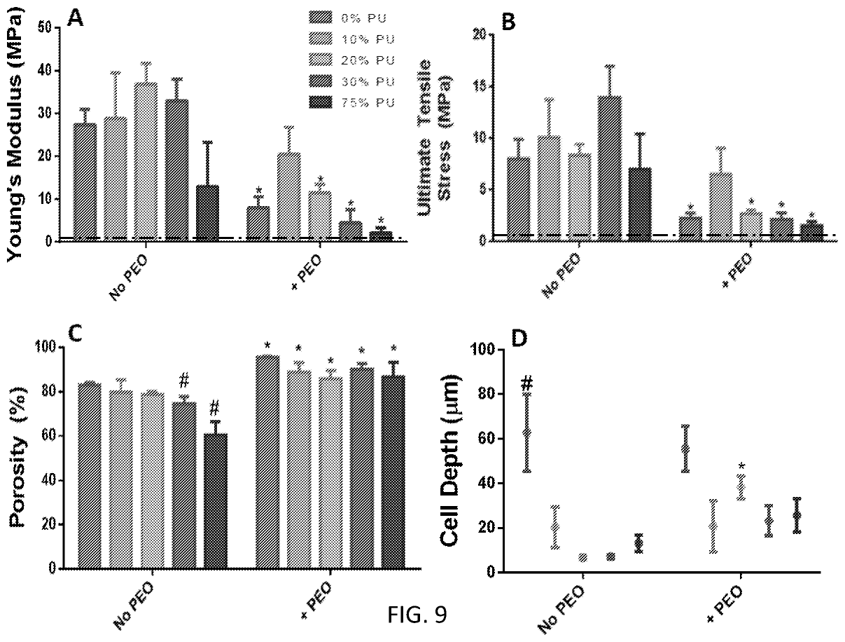

FIGS. 9A-D are graphs showing changes in experimental results from adding PEO to the scaffold. Data is presented as mean ±SD. A single asterisk (*) represents significantly different from same composite without PEO (p<0.5), while a single hashtag (#) represents significantly different from all other groups at same PEO content (p<0.5). FIG. 9A shows a graph depicting changes in Young's Modulus of the five materials without (left; No PEO) and with (right; +PEO) the addition of PEO to the scaffold. The dash-dot line shows Young's Modulus of control scaffolds of porcine heart tissue (e.g., at about 0.05 MPa or 50 kPa).

As demonstrated by the graph of Fif. 9A, the addition of PEO significantly lowered the Young's Modulus for scaffolds fabricated from each PU formulations, except the 10% PU group (orange bar; p<0.05). The modulus of the 75% PU+PEO material was 2.19 MPa, making it the group closest to the control modulus of porcine ventricular tissue (0.05 MPa), but the Young's Modulous of scaffolds from this group was not significantly different those of the other groups (i.e. 0%, 20%, or 30% PU+PEO).

FIG. 9B shows a graph depicting changes in ultimate tensile stress with the addition of PEO to the scaffold. The dash-dot line shows the ultimate stress of scaffolds with porcine heart tissue as a control (e.g., at about 0.130 MPa or 130 kPa). As shown, the addition of PEO lowered ultimate tensile stress significantly in all PU formulations except for the 10% PU group (p<0.05). Though 1.56 MPa for the 75% PU+PEO group was the closest to the ultimate stress for the control porcine ventricular tissue, this group was not significantly different from the 0%, 20%, and 30% PU groups with PEO (p>0.05). All PU formulations, with and without PEO, withstood enough stress to hold up to ventricular pressures measured around 0.2 MPa or 200 kPa.4.

FIG. 9C shows a graph depicting changes in porosity with the addition of PEO to the scaffold. As shown, without PEO, the higher PU concentration groups, 30% PU and 75% PU, had significantly lower porosity (p<0.05). The addition of PEO increased the porosity of all PU formulations (p<0.05). Given that porosity was increased in all PEO-inclusive formulations, cell infiltration was expected to also increase in these groups.

FIG. 9D shows a graph depicting changes in cell depth with the addition of PEO to the scaffold (e.g., the depth of hDF infiltration into scaffolds). As shown, though the addition of PEO increased the depth of cell infiltration in the 20%-75% PU+PEO groups, the increase was only significant in the 20% PU+PEO group versus the 20% PU group with no PEO (p<0.05). The 0% PU groups, both with and without PEO, had the largest depth of cell infiltration. Scaffold shrinkage, as a quantification of pore collapse, was observed in all PEO-inclusive formulations, but especially in formulations with higher PU content. This shrinkage could explain the reduced cell infiltration despite increased porosity in higher PU content formulations.

As depicted in FIGS. 9A-9D, although the 75% PU+PEO group demonstrated mechanical properties closest to native heart tissue, this group did not have high cell infiltration depth. The 0% PU+PEO group had high cell infiltration depth and had mechanical properties that were not significantly different from the 75% PU+PEO group.

### Example 7—Rat Right Ventricle Replacement Model

In this study, we fabricated a prevascularized BPUR reinforced PEG-fibrin patch as presently disclosed, for example by seeding human umbilical endothelial cells (HUVECs) and human c-Kit+amniotic fluid stem cells (AFSCs). The disclosed patch was tested in an athymic nude rat right ventricle wall defect replacement model. Heart function was measured, and histologic sections were evaluated for fibrosis, macrophage infiltration, vascularization and muscularization at 2 months postsurgery. As shown below, the prevascularized cardiac patch would induce better muscular and vascular ingrowth, resulting in improved heart function over a non-cell seeded patch.

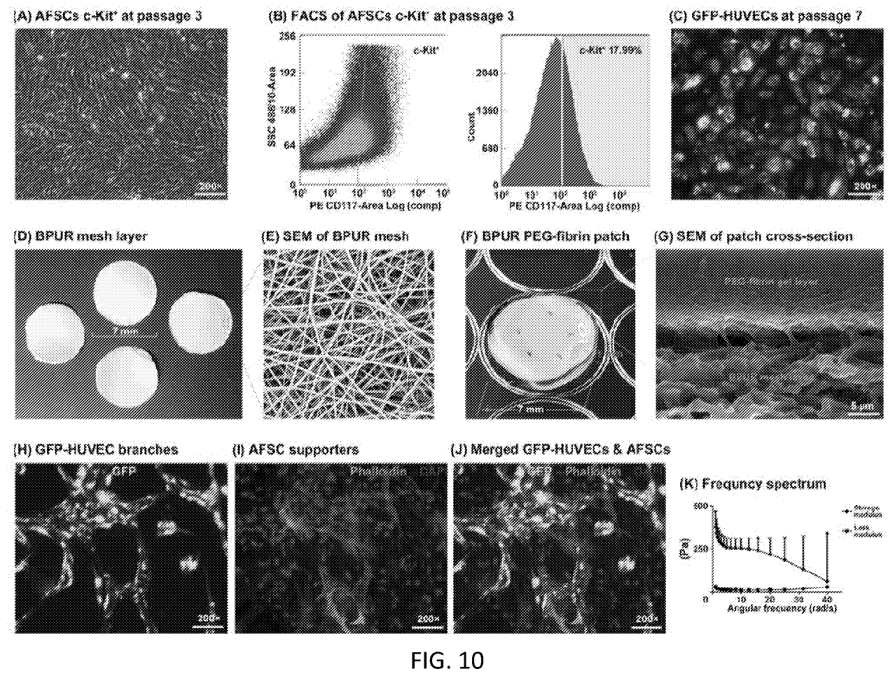

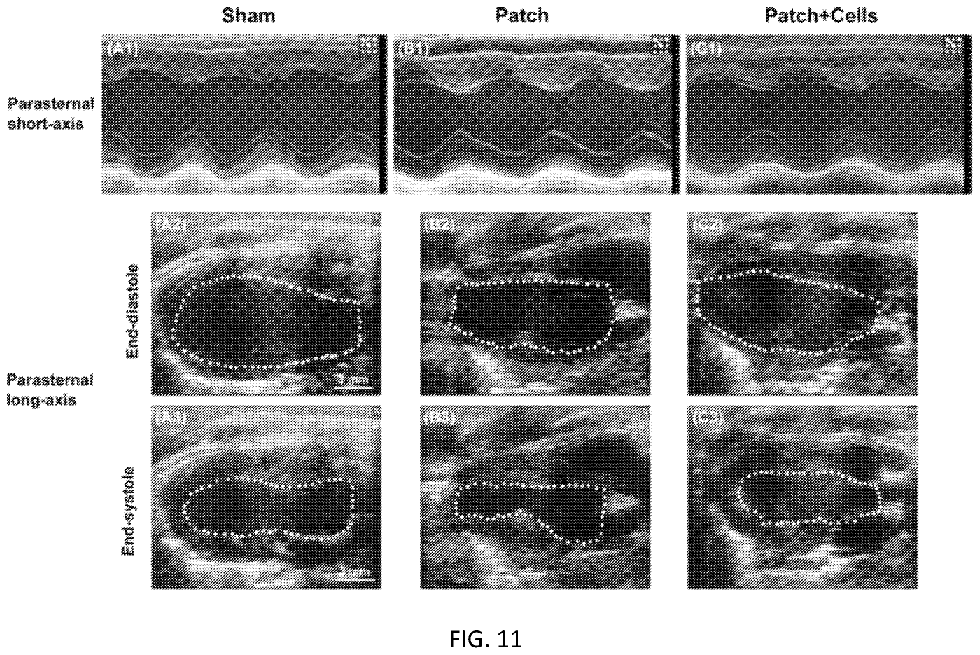

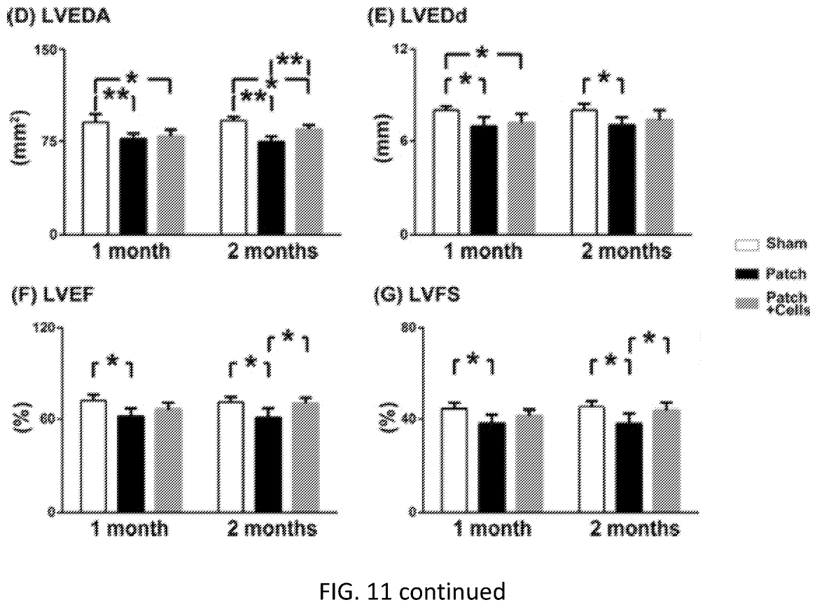

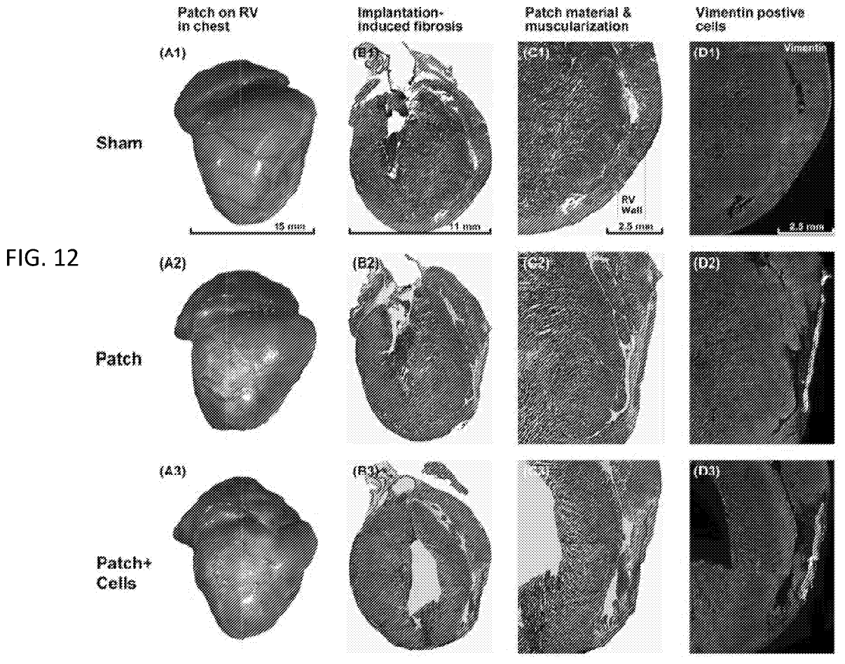

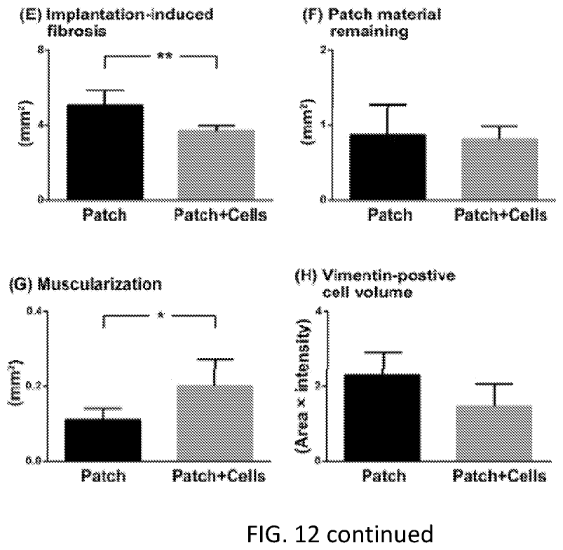

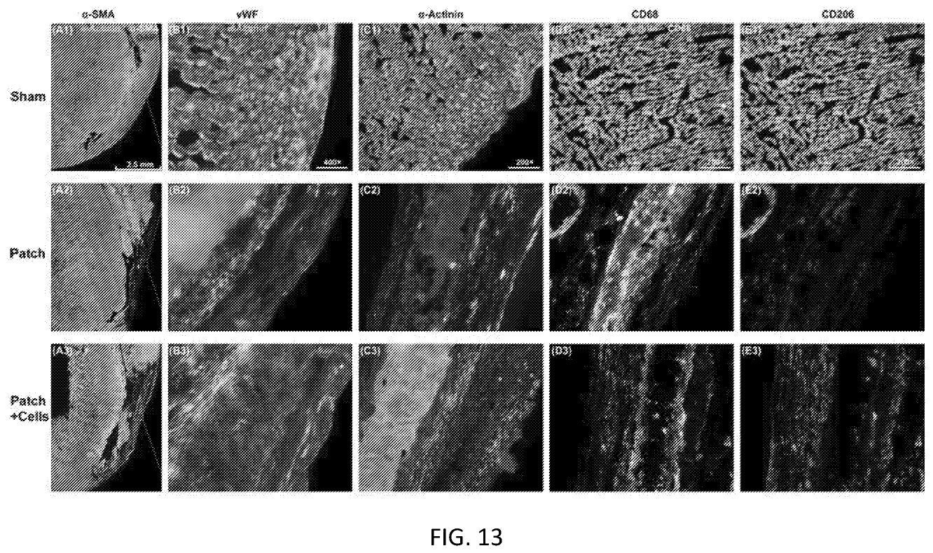

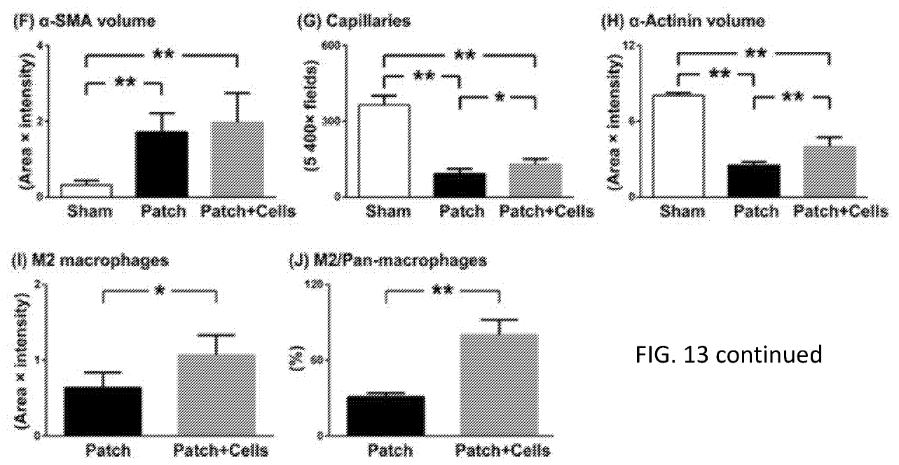

As shown in FIG. 10, the presently disclosed reinforced fibrin gel patches (1 mm in thickness and 7 mm in diameter) were fabricated by mixing 30 μl PEGylated human fibrinogen (40 mg/ml) with 15 μl PBS containing 80 U/ml human thrombin plus 15 μl cell suspension containing GFP-HUVECs (4×10⁶/ml)/human c-Kit+AFSCs (4×10⁶/ml) (4:1) or 15 μl PBS, and reinforced with a BPUR layer (80 μm in thickness and 7 mm in diameter). These patches were cultured in an incubator with GEM-2 media for 3 days before implantation. Twenty male athymic nude rats (NIH-Foxn1^{rnu}) weighing 400 ±20 g were randomly assigned to a sham group (n=5), patch group (n=7) and patch+cells group (n=8). Defects of 2-3 mm in diameter were created through the right ventricle free wall and patches were sutured as a myocardial wall replacement, as previously described. During the surgery, 0, 2, and 2 animals died in the sham, patch and patch+cells groups respectively. Echocardiography was performed at 1- and 2-month time points, and electrocardiogram signals were recorded for 30 min at the 2-month endpoint. Histology was used to measure fibrosis and patch material remaining; immunofluorescence staining was used to evaluate vascularization, muscularization and macrophage infiltration.

The Young's modulus of the PEG-fibrin was 713±226 Pa (n=5). BPUR fiber meshes had an average fiber diameter of 1.5±0.8 μm (n=5) and an average tensile modulus of 2.9±0.4 MPa (n=5). All animals surviving the surgery survived to the 2-month endpoint. One animal in the shame group had sporadic atrial premature beats (APBs) (1/5), one animal in the patch group had frequent ventricular beats (1/5) and one animal in the patch+cells group had sporadic APBs (¹/₆).

In this study, we found that a prevascularized fibrin gel patch induced less fibrosis and material remaining, better vascularization and muscularization, larger amount of M2 macrophage infiltration, and improved heart function compared witha non-cell seeded patch at 2 months post-surgery. Surgical correction of CHDs relies on biomaterials that are feasible, biocompatible, prone to endothelialization, disposed to remodeling and integration, and functionally long-lasting. In this study, HUVECs and c-Kit+AFSCs were cultured inside PEG-fibrin gel and at 3 days they successfully formed vascular branches before implantation (FIG. 10 Panels H-J). Fibrin could be produced autologously from patient's blood and it plays critical roles in blood clotting, cell-matrix interaction, inflammation, and wound healing; incorporation of PEG into fibrin dramatically increased scaffold stiffness and stability. Biodegradable polyurethane formations have been used in cardiac tissues owing to their excellent biocompatibility and hemocompatibility, and their mechanical properties can be controlled by modifying the chemical structure. We were unable to trace the seeded GFP expressing HUVECs and human c-Kit+ AFSCs from the consecutive sections in the patched area at 2 months post-surgery. We postulate that the beneficial effects of the cell seeded patch on vascularization, muscularization, and improved heart function could be due to paracrine effects.

While multiple embodiments are disclosed, still other embodiments of the present invention will become apparent to those skilled in the art from the detailed description. The invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

We claim:

1. A heart patch device comprising;
a biodegradable polymeric mesh scaffold; and
a biodegradable gel.

2. The heart patch device of claim 1, wherein the polymeric mesh scaffold is made of a material comprising one or more of gelatin, polyurethane, and polycaprolactone.

3. The heart patch device of any of claim 1 or 2, wherein the gel comprises one or more of fibrin and polyethylene glycol.

4. The heart patch device of any of claims 1-3, wherein the gel comprises fibrinogen.

5. The heart patch device of any of claims 1-4, wherein the gel comprises fibrinogen with one or more polyethylene glycol molecules.

6. The heart patch device of any of claims 1-5, wherein the gel further comprises thrombin.

7. The heart patch device of any of claims 1-6, wherein the polymeric mesh scaffold comprises biodegradable polyurethane.

8. The heart patch device of any of claims 1-7, further comprising one or more cells selected from a stem cell, an endothelial cell, and mixtures thereof.

9. The heart patch device of any of claims 1-8, further comprising one or more of umbilical vein endothelial cells and induced stem cells.

10. A method for making the heart patch device, comprising;
forming a mesh scaffold by electrospinning, wherein the mesh scaffold comprises a biodegradable polymer;
forming a gel layer, wherein the gel is biodegradable; and
combining the mesh scaffold with the gel layer.

11. The method of claim 10, wherein additional gel is added to the mesh scaffold after it is adjacent the gel layer.

12. The method of claim 10 or claim 11, wherein the polymeric mesh scaffold is electrospun in the presence of polycaprolactone.

13. The method of any of claims 10-12, wherein the gel comprises one or more of fibrin and polyethylene glycol.

14. The method of any of claims 10-13, wherein the gel comprises fibrinogen.

15. The method of any of claims 10-14, wherein the gel comprises polyethylene glycol molecules conjugated to fibrinogen.

16. The method of any of claims 10-15, wherein the gel further comprises thrombin.

17. The method of any of claims 10-16, wherein the polymeric mesh scaffold comprises biodegradable polyurethane.

18. The method of any of claims 10-17, wherein one or more cells selected from a stem cell, an endothelial cell, and mixtures thereof are added to the gel before combining with the mesh scaffold.

19. The method of any of claims 10-18, wherein one or more of umbilical vein endothelial cells and induced stem cells are added to the gel before combining with the mesh scaffold.

20. The method of any of claims 10-17, wherein one or more cells selected from a stem cell, an endothelial cell, and mixtures thereof are added to the patch after combining the gel with the mesh scaffold.

21. The method of any of claims 10-18, wherein one or more of umbilical vein endothelial cells and induced stem cells are added to the patch after combining the gel with the mesh scaffold.

22. The method of any of claims 18-21, wherein the patch is incubated in culture media for from 0 to 336 hours.

23. A method of treating a subject having a heart defect, the method comprising:
obtaining a heart patch device, the heart patch device comprising
a biodegradable polymeric mesh scaffold; and
a biodegradable gel
placing the heart patch device at or near the heart defect;
connecting the heart patch device to the heart with one or more sutures; and thereby treating the subject having the heart defect.

24. The methods of claim 23, wherein the heart patch device includes one or more mammalian cells.

25. The methods of claim 23 or 24, wherein the heart patch device includes one or more autologous cells.

26. The methods of any of claims 23-25, wherein the heart patch device includes one or more non-autologous cells.

27. The methods of any of claims 23-26, wherein the heart patch device includes one or more cells expressing one or more of CD31 (PECAM1), CD144 (VE-Cadherin), and CD309 (VEGFR2).

28. The methods of any of claims 23-27, wherein the heart patch device includes one or more stem cells.

29. The methods of claim 28, wherein the stem cells are induced stem cells.

30. The method of any of claims 23-29, wherein the gel comprises one or more of fibrin and polyethylene glycol.

31. The method of any of claims 23-30, wherein the gel comprises fibrinogen.

32. The method of any of claims 23-31, wherein the gel comprises polyethylene glycol molecules conjugated to fibrinogen.

33. The method of any of claims 23-32, wherein the gel further comprises thrombin.

34. The method of any of claims 23-33, wherein the polymeric mesh scaffold comprises biodegradable polyurethane.

35. The method of any of claims 23-34, wherein the heart patch device comprises one or more of umbilical vein endothelial cells and induced stem cells.

36. The method of any of claims 23-35, wherein the patch is incubated in culture media for from 0 to 336 hours.
